(19) United States
(12) Patent Application Publication
(10) Pub. No.: US 2026/0315722 A1
(43) Pub. Date: Oct. 15, 2026

(54) DIGITALLY ENABLED, PRESSURE SWING ADSORPTION-BASED INTERMITTENT HYPOXIA-HYPEROXIA TRAINING SYSTEMS

(71) Applicant: L-Newco, Inc. - DBA Vitaliti, Hillsborough, CA (US)

(72) Inventors: Denis Varvanets, Los Gatos, CA (US); Stephen Price, Spring, TX (US); Mahmood Panjwani, Hillsborough, CA (US); Ganga Nadella, Menlo Park, CA (US); Danyal Panjwani, Hillsborough, CA (US); Zhou Xingyuan, Shenyang City (CN)

(73) Assignee: L-Newco, Inc. - DBA Vitaliti, Hillsborough, CA (US)

(21) Appl. No.: 19/717,230

(22) Filed: Jun. 23, 2026

Related U.S. Application Data
(63) Continuation-in-part of application No. 19/304,356, filed on Aug. 19, 2025.
(60) Provisional application No. 63/684,748, filed on Aug. 19, 2024.

(51) Int. Cl. A61M 16/10; A61M 16/20

(52) U.S. Cl. CPC A61M 16/101; A61M 16/20

(57) ABSTRACT

A device for intermittent hypoxia-hyperoxia training includes a main chassis; a pressure swing adsorption (PSA) system housed within the chassis, the PSA system configured to generate a hyperoxic gas stream and a hypoxic gas stream; a buffering reservoir housed within the main chassis and configured to store the hypoxic gas stream; a first valve coupled to the hyperoxic gas stream; a second valve coupled to the buffering reservoir; a delivery path coupled to the first valve and the second valve, the delivery path including an output port; and a blower coupled to the delivery path, the blower being operable between an activated state and a deactivated state, the blower providing an ambient air stream to the delivery path when in the activated state.

### CROSS-REFERENCE TO RELATED APPLICATIONS

This application is a Continuation-in-Part of U.S. patent application Ser. No. 19/304,356, filed Aug. 19, 2025, entitled “Digitally Enabled, Pressure Swing Adsorption-Based Intermittent Hypoxia-Hyperoxia Training Systems” which claims priority to U.S. Provisional Patent Application No. 63/684,748, filed Aug. 19, 2024, entitled “AI-Powered Intermittent Hypoxia-Hyperoxia Training Device,” the entire contents of which are incorporated by reference herein.

### TECHNICAL FIELD

The present disclosure generally relates to intermittent hypoxia-hyperoxia training (IHHT) systems, and more specifically, to neurovascularly guided control of IHHT training using multi-modal physiological sensing, coupled to a zeolite-based digitally controlled pressure swing adsorption system with a single-mask output and an integrated, internal gas buffer.

### BACKGROUND OF THE DISCLOSURE

Intermittent hypoxia-hyperoxia training (IHHT) alternates reduced-oxygen (hypoxia) and elevated-oxygen (hyperoxia) breathing periods to elicit adaptations in mitochondrial efficiency, autonomic balance, and cardiorespiratory performance. Conventional IHHT implementations typically follow preprogrammed schedules (e.g., fixed-length hypoxic bouts followed by fixed recovery periods) that are not personalized to a user's moment-to-moment physiological state. In such systems, the delivered dose is primarily defined by time at a target gas composition rather than by the user's actual physiological response.

Existing control strategies often rely on a single parameter, most commonly peripheral blood oxygen saturation (SpO₂), to gate or terminate hypoxic intervals. While SpO₂ is useful, it can exhibit lag relative to arterial oxygen dynamics and may not reflect neuro-autonomic load or metabolic stress during or after a hypoxic exposure. As a result, two individuals with identical SpO₂ traces can experience markedly different autonomic activation and glycemic excursions, leading to inconsistent outcomes and potential safety concerns when using uniform protocols.

From a hardware perspective, many IHHT devices generate one conditioned gas stream and blend with ambient air to approximate hypoxic or hyperoxic conditions, or they rely on membrane-based separation that limits achievable hyperoxic purity. Transitions between gas states can be slow, and some systems require users to swap masks or external hoses when switching phases. To smooth flow, a number of devices employ external inflatable buffer bags that increase footprint, introduce potential leak/failure points, complicate sanitation, and add compliance that can degrade the responsiveness of closed-loop control.

These architectural choices create specific technical constraints: (i) limited peak fraction of inspiration O₂ (FiO₂) for short, protective hyperoxic bursts; (ii) sluggish phase switching that reduces the precision of dose delivery; and (iii) transport and compliance delays introduced by long external circuits and bags, which blur the temporal alignment between sensor-detected events and controller actions. Due to the sluggish phase switching, transport and compliance delays, and other technical constraints, dynamic control of the IHHT devices during treatments becomes infeasible or extremely challenging.

Further, personalization remains underdeveloped in the existing IHHT devices. Real-time autonomic markers such as heart-rate variability (HRV) provide insight into sympathetic/parasympathetic balance, blood pressure (BP) is the hemodynamic guardrail, perfusion index (PI) is both a signal-quality sentinel and a physiologic window into peripheral vasomotor tone, while continuous glucose monitoring (CGM) reflects metabolic stress and recovery kinetics. Conventional systems generally do not fuse these signals, and therefore cannot obtain a live gauge of how hard the nervous system is working to cope with the stimulus, and cannot enforce individualized “metabolic guardrails,” such as limiting hypoxic dose when glucose excursion or recovery slope exceeds user-specific thresholds. Data fusion for IHHT presents its own challenges: signals arrive at different cadences and latencies (e.g., interstitial glucose lag relative to blood, HRV requiring windowed estimation, motion artifacts affecting photoplethysmography).

Furthermore, many existing IHHT devices are not intended for personal home use as they require specialized equipment, trained operators, are intended to serve multiple patients in a hospital or clinical setting, or are too bulky to transport and store in a home setting. For example, many existing IHHT devices utilize external buffering for hypoxic and hyperoxic gas streams requiring accessory bags and tubing, multi-hose or multi-mask patient interfaces requiring manual management, and lack gas composition control.

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY OF THE DISCLOSURE

The present disclosure addresses the above-mentioned problems and other problems in the existing IHHT devices by providing artificial intelligence (AI)-powered IHHT devices with protocols that can be personalized in advance and adapted in actual training processes.

In one aspect, an apparatus for intermittent hypoxia-hyperoxia training includes: a main chassis; a pressure swing adsorption (PSA) system housed within the chassis, the PSA system configured to generate a hyperoxic gas output and a hypoxic gas output; a buffering reservoir housed within the main chassis, configured to receive the hypoxic gas output from the PSA system; a single user output port; a valve mechanism having a first inlet connected to an output of the buffering reservoir, a second inlet connected to the hyperoxic gas output of the PSA system, and an outlet connected to the single user output port; and a computation unit configured to control the valve mechanism, thereby selecting whether hypoxic gas from the buffering reservoir or hyperoxic gas from the PSA system is delivered to the single user output port.

In another aspect, a method, for providing adaptive IHHT training using the apparatus described elsewhere, includes: receiving, at the external user device, a plurality of real-time physiological data streams from one or more wearable sensors monitoring a user; processing, by the external user device, said physiological data streams to determine a need for a protocol adjustment; transmitting, from the external user device to the computation unit of the apparatus via the wireless transceiver, a command to implement the protocol adjustment; and executing, by the computation unit, the command to adjust the training being administered to the user.

In another aspect, a system for IHHT training includes: one or more sensors configured to obtain physiological data of a user under the IHHT training; a computation unit configured to compute a score based on the physiological data of the user; a gas generator produce a nitrogen-enriched, oxygen-reduced hypoxic gas stream and an oxygen-enriched hyperoxic gas stream, where the gas generator is further fluidly connected to a first buffering reservoir for buffering the hyperoxic gas stream and a second buffering reservoir for buffering the hypoxic gas stream; a computer-controlled switching valve fluidically coupled to the first buffering reservoir and the second reservoir; and a single breathing interface coupled to the switching valve, where the computation unit further includes a controller operatively coupled to the switching valve to adjust a flow of at least one of the hyperoxic gas stream or the hypoxic gas stream during a training session responsive to the generated score.

In another aspect, an IHHT device includes: a main chassis, a a pressure swing adsorption (PSA) system housed within the main chassis, the PSA system configured to generate a hyperoxic gas stream and a hypoxic gas stream; a buffering reservoir housed within the main chassis and configured to store the hypoxic gas stream; a first valve coupled to the hyperoxic gas stream; a second valve coupled to the buffering reservoir; a delivery path coupled to the first valve and the second valve, the delivery path including an output port; and a blower coupled to the delivery path, the blower being operable between an activated state and a deactivated state, the blower providing an ambient air stream to the delivery path when in the activated state.

In another aspect, a method includes receiving, at a remote computing device, user data; determining, by a processing circuit of the remote computing device, a treatment protocol based on the user data, the treatment protocol including a hypoxic protocol and a recovery protocol; transmitting, by the remote computing device, a first signal commanding execution of the hypoxic protocol to an intermittent hypoxia-hyperoxia training (IHHT) device; receiving, at the remote computing device, sensor data from one or more physiological sensors; determining, by the processing circuit, an adjustment to the hypoxic protocol based on the sensor data; and transmitting, by the remote computing device, a second signal commanding execution of the recovery protocol to the intermittent hypoxia-hyperoxia training (IHHT) device.

In another aspect a system includes: an intermittent hypoxia-hyperoxia training (IHHT) device and a remote computing device. The IHHT device includes: a pressure swing adsorption (PSA) system configured to generate an oxygen-enriched gas and an oxygen-reduced gas; a delivery path; and a valve assembly coupled to the PSA system and the delivery path, the valve assembly configured to selectively produce a hypoxic gas stream or a hyperoxic gas stream. The remote computing device is configured to: determine a treatment protocol; and transmit control signals to the IHHT device to control operation of the PSA system and the valve assembly according to the treatment protocol.

The foregoing is a summary and thus contains, by necessity, simplifications, generalizations, and omissions of detail; consequently, the summary is illustrative only and is not limiting in any way. Other aspects, inventive features, and advantages of the systems and/or processes described herein may become apparent in the non-limiting detailed description set forth herein.

### BRIEF DESCRIPTION OF THE DRAWINGS

These and other features, aspects, and advantages of the present disclosure may become better understood with regard to the following description, and accompanying drawings, where:

FIG. 1 is a schematic diagram of an AI-powered intermittent hypoxia-hyperoxia training device, in accordance with some embodiments.

FIG. 2 shows example system components for an AI-powered intermittent hypoxia-hyperoxia training device, in accordance with some embodiments.

FIG. 3 shows an example digitally controlled pressure swing adsorption system inside an AI-powered intermittent hypoxia-hyperoxia training device, in accordance with some embodiments.

FIG. 4 illustrates an example control of an AI-powered intermittent hypoxia-hyperoxia training device through a mobile device, in accordance with some embodiments.

FIG. 5 shows an example accordion-style reservoir for an internal buffering reservoir of an AI-powered intermittent hypoxia-hyperoxia training device, in accordance with some embodiments.

FIG. 6 shows an example process flow for an AI-powered intermittent hypoxia-hyperoxia training session, in accordance with some embodiments.

FIG. 7 shows an example computing device for implementing systems and methods described in reference to FIGS. 1-6.

FIG. 8 shows an example intermittent hypoxia-hyperoxia system, according to some embodiments.

FIG. 9 shows an example intermittent hypoxia-hyperoxia training device, in accordance with some embodiments.

FIG. 10 shows a portion of the intermittent hypoxia-hyperoxia training device of FIG. 9, in accordance with some embodiments.

FIG. 11 shows a portion of the intermittent hypoxia-hyperoxia training device of FIG. 9, in accordance with some embodiments.

FIG. 12 shows an example gas delivery system of an intermittent hypoxia-hyperoxia training device, in accordance with some embodiments.

FIG. 13 shows an example of a main board of an intermittent hypoxia-hyperoxia training device, in accordance with some embodiments.

FIG. 14 shows an example of a method for controlling an intermittent hypoxia-hyperoxia training device, in accordance with some embodiments.

FIG. 15 shows an example of a method for controlling a blower of an intermittent hypoxia-hyperoxia training device, in accordance with some embodiments.

FIG. 16 shows an example of another method for controlling an intermittent hypoxia-hyperoxia training device, in accordance with some embodiments.

FIG. 17 shows an example of a method for dynamically controlling an intermittent hypoxia-hyperoxia training device, in accordance with some embodiments.

FIG. 18 shows an example of a method for determining a treatment protocol for an intermittent hypoxia-hyperoxia training device, in accordance with some embodiments.

### DETAILED DESCRIPTION

To make the aforementioned objects, features, and advantages of the present disclosure more obvious and understandable, the present disclosure may be further described below with reference to the accompanying drawings and embodiments.

It should be noted that specific details are set forth in the following description to fully understand the present disclosure. However, the present disclosure may be implemented in many other ways different from those described herein, and those skilled in the art may make similar generalizations without violating the connotation of the present disclosure. Therefore, the present disclosure is not limited by the specific embodiments disclosed below.

The terms used in the embodiments of the present disclosure are only for the purpose of describing specific embodiments and are not intended to limit the present disclosure. The singular forms of “a”, “said”, and “the” used in the embodiments of the present disclosure and the appended claims are also intended to include plural forms unless the context clearly indicates other meanings.

It should be noted that the example embodiments may be implemented in various forms, and should not be construed as being limited to the embodiments set forth herein. On the contrary, the provision of these embodiments makes the present disclosure more comprehensive and complete, and fully conveys the concept of the example embodiments to those skilled in the art. The same reference numerals in the figures indicate the same or similar structures, and thus their repeated description may be omitted. In addition, the similarities between the embodiments may not be repeated.

### Motivation and Benefits

Heart failure is a leading cause of morbidity, mortality, and healthcare expenditure, affecting an estimated 6.7 million adults in the United States alone, with approximately one million new diagnoses annually. Cardiac rehabilitation, a structured program of monitored exercise, risk factor management, and patient education, is a Class I recommendation by the American Heart Association (AHA) and the American College of Cardiology (ACC) for patients following myocardial infarction, coronary artery bypass grafting, percutaneous coronary intervention, valve repair or replacement, and stable heart failure. Clinical evidence demonstrates that completion of cardiac rehabilitation is associated with a 25-30% reduction in cardiovascular mortality and significant improvements in functional capacity, quality of life, and hospital readmission rates.

Despite this evidence, cardiac rehabilitation completion rates remain critically low. Of eligible patients, only 20-30% receive a referral; of those referred, only 25-35% complete the prescribed program. The net result is that approximately 5-10% of eligible patients receive the full benefit of cardiac rehabilitation.

Barriers to cardiac rehabilitation adherence include kinesiophobia, travel burden, physical limitation, scheduling conflicts, and psychological barriers. Kinesiophobia, the fear that physical exertion will trigger a cardiac event, is documented in 40-60% of cardiac patients and is the single strongest psychological predictor of rehabilitation dropout. Travel burden is a primary logistical barrier as the average cardiac rehabilitation facility is located more than 30 minutes from a patient's home, with rural patients facing 60 minutes or more of one-way travel with programs typically requiring three visits per week for 12 weeks. Physical limitation prevents participation for many patients with advanced heart failure (NYHA Class III-IV) who are physically unable to perform standard exercise protocols at the intensity required for cardiovascular conditioning. Scheduling conflicts arise because facility-based sessions occur during business hours and require dedicated transport. Psychological barriers including depression (comorbid in 20-40% of heart failure patients), anxiety, and low self-efficacy further reduce motivation and follow-through.

IHHT offers a mechanistically distinct approach to rehabilitation. IHHT produces physiological adaptations overlapping substantially with those of aerobic exercise including improved mitochondrial biogenesis, enhanced oxidative phosphorylation efficiency, autonomic rebalancing with increased vagal tone, erythropoietin-mediated improvements in oxygen-carrying capacity, and vascular endothelial adaptation through controlled alternation of hypoxic and hyperoxic breathing that requires no physical exertion. IHHT functions as an exercise mimetic by delivering a subset of the conditioning benefits of exercise through passive respiratory stimulus, eliminating kinesiophobia (no exercise is involved), travel burden (when delivered at home), physical limitation (the therapy is entirely passive), and scheduling constraints (self-administered sessions at times of the patient's choosing).

The disclosure is motivated by the need to deliver IHHT in a way that is (i) mechanically capable of rapid, precise transitions without bulky external components enabling at-home administration, and (ii) physiologically individualized in real time. Legacy hardware (single stream with blending, external buffer bags) slows transitions, increases leakage/failure points, and complicates sanitation and transport, which makes the conventional time-based protocols and single-parameter control yield variable outcomes and safety tradeoffs.

The benefits of the disclosed system follow from a hardware update that enables responsiveness that matches the temporal resolution of the control logic. A digitally controlled pressure swing adsorption (PSA) system and a fast switching valve produce both hypoxic and high-purity hyperoxic streams to a single breathing interface (mask) and transition between them with minimal delay. An internal buffer reduces transport and compliance lag relative to external bags and long circuits, improving the alignment between sensed physiological events and delivered gas composition and thereby sharpening the effective dose profile.

By computing a unified neurovascular and/or stress score from SpO₂, HRV, blood pressure, perfusion index, and CGM, the controller also titrates hypoxic and hyperoxic intervals to the individual in real time due to the updated hardware, reducing both under- and over-dosing and stabilizing outcomes across sessions. Personalized “metabolic guardrails”, such as limiting hypoxic exposure when glucose excursions or recovery slope exceed user-specific thresholds, further improve safety while preserving therapeutic effect, and automatic hyperoxic rescue bursts provide additional protection when the model detects excessive load.

Usability and reliability are enhanced by routing both phases through a single patient interface and by integrating the buffer within the chassis. Eliminating external inflatable bags reduces footprint and setup steps, removes common leak and failure points, and simplifies cleaning and sanitation, all of which support consistent field operation in clinical and wellness settings.

The system also produces measurable, longitudinal biomarkers to guide programming. A standardized hypoxic challenge paired with CGM excursion and recovery kinetics yields a biomarker of metabolic flexibility that can inform clinician oversight and program tuning. Wireless sensor ingress and cloud reporting support remote monitoring and software updates, while faster transitions reduce session length and staff interventions. The zeolite-based architecture avoids membrane supply constraints and lowers consumables, improving deployment economics.

It is to be noted that the benefits and advantages described herein are not all-inclusive, and many additional features and advantages will be further described under the context of specific embodiments. In addition, some additional features and advantages will become apparent to one of ordinary skill in the art in view of the figures and the following descriptions.

### Overall System

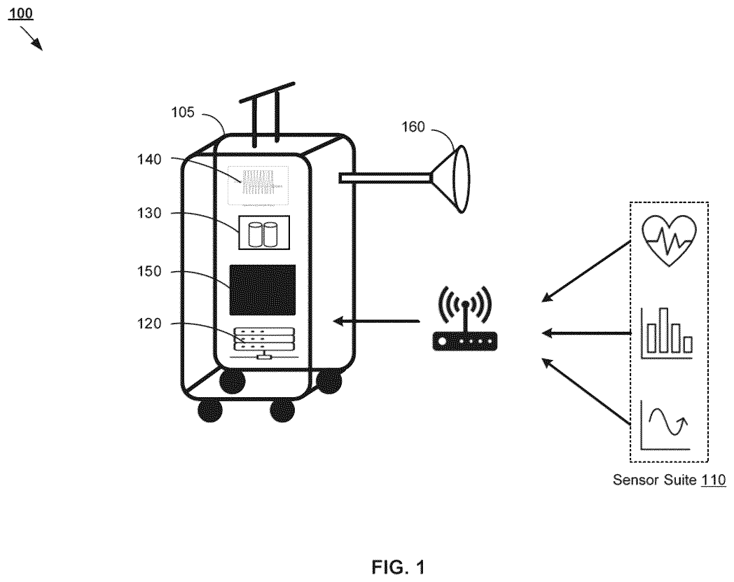

In the following, an AI-powered IHHT device 100 is described with reference to specific hardware components and/or software components. As illustrated in FIG. 1, exemplary hardware components may include: (a) a sensor suite 110 including one or more of SpO₂ sensor, an HRV sensor (e.g., electrocardiogram (ECG) or photoplethysmography (PPG)-derived), a blood pressor sensor, a CGM sensor, or other different physiological sensors; (b) a computation unit 120 with processors, memory, and storage executing a trained model included therein; (c) a digitally controlled PSA system 130; (d) a buffering reservoir 140 comprising an internal, chassis-integrated reservoir (which itself may be a part of the digitally controlled PSA system); (e) a controller 150 driving valves, flow, and FiO₂; and wireless interfaces (e.g., Bluetooth, ANT+) (not shown) for sensor ingress and/so for possible other wireless connections, such as connecting to a user's mobile phone or a remote monitoring device. All of these components are internal components inside a chassis 105, where a single breathing interface (mask or nasal interface) 160 is connected. In some embodiments, the IHHT device 100 may further include or be coupled to a mobile and/or local UI, secure storage, and cloud services for remote monitoring, clinician dashboards, and model updates. In some embodiments, the IHHT device 100 is packaged in a carry-on-sized, wheeled chassis 105 with a retractable handle and a total mass under a certain value (e.g., under 10 kg, 15 kg, 20 kg, 30 kg, 40 kg, 50 kg, 60 kg, etc.), enabling one-handed transport like a suitcase between rooms or sites.

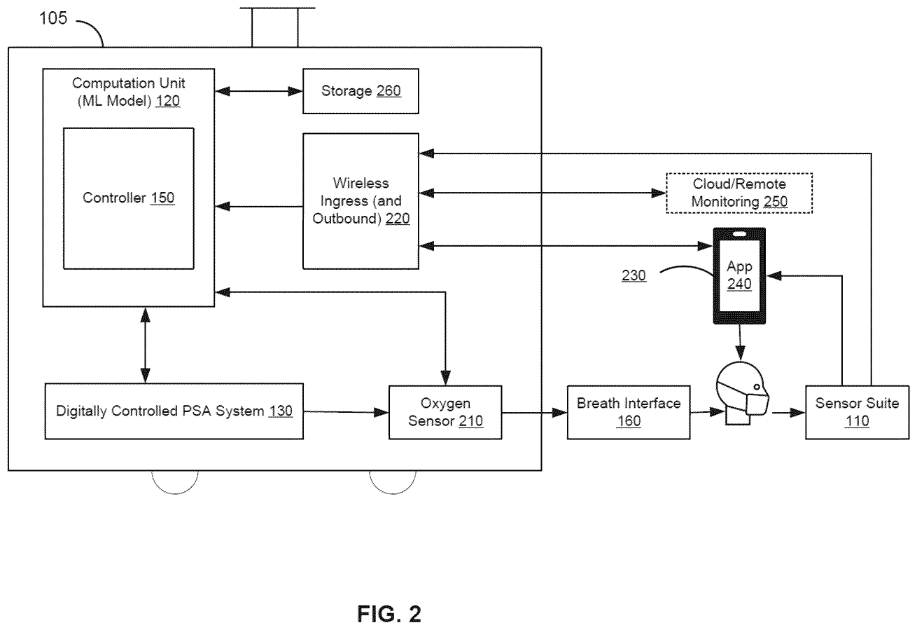

In operation, briefly, the digitally controlled PSA system 130 may provide necessary oxygen levels, which are provided to the breathing interface 160 through the buffering reservoir 140 that smooths flow to the breathing interface, while safety monitors enforce guardrails. At the same time, sensor data from the sensor suite 110 is collected, which then arrives at the computation unit 120/controller 150 via wireless ingress, which drives the controller 150 to dynamically adjust FiO₂ targets and interval timing. For example, the controller 150 may actuate the switching valve to select between the hypoxic gas stream and the hyperoxic gas stream provided by the PSA system 130. The operation of the disclosed IHHT device 100 is further described in detail with reference to specific components in FIG. 2.

### Specific Components in the IHHT Device

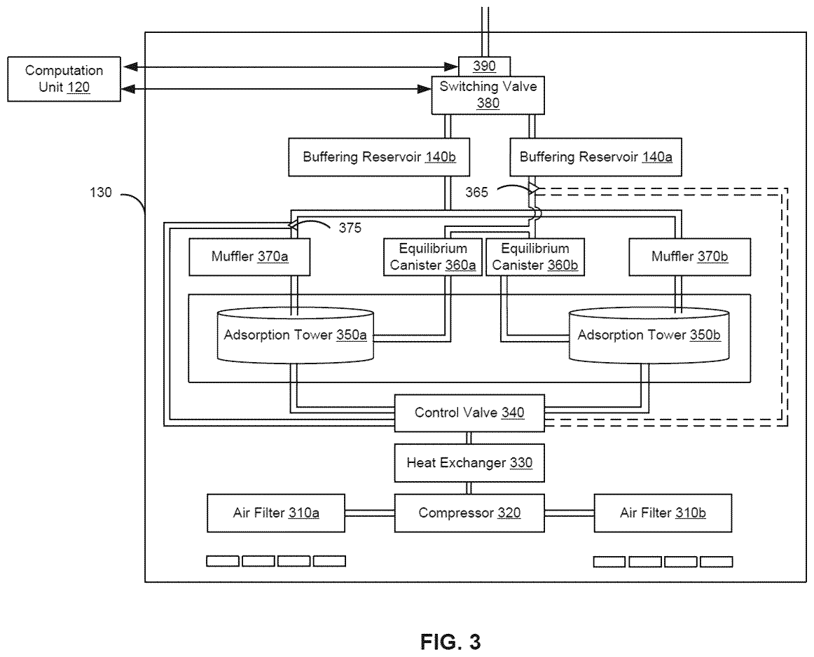

The digitally controlled PSA system 130 may condition ambient air (filtration and compression) and perform gas separation (e.g., via pressure-swing adsorption and/or controlled dilution) to produce a nitrogen-enriched, oxygen-reduced stream at a commanded FiO₂ for hypoxic phases (also referred to as hypoxic gas stream), as well as an oxygen-enriched stream at a commanded purity (e.g., FiO₂ at 80% or higher) (also referred to as hyperoxic gas stream). It may regulate gas delivery using a product volume to stabilize flow and pressure, verify composition with an oxygen (or proxy) sensor 210, and isolate its path with one-way valving to prevent cross-flow. In some embodiments, certain control interfaces may be further included to allow the controller 150 to set and/or further adjust targets, ramps, and limits (e.g., FiO₂, cycle timing, purge ratio). In some embodiments, safety logic may be further included to enforce guardrails (e.g., minimum breathable state on fault, leak/occlusion detection) and bias the circuit to a safe condition upon error or power loss, before handing the hypoxic output to the blender/switching stage. The specific details for the digitally controlled PSA system are further described in FIG. 3.

The oxygen sensor 210 is disposed between the buffering reservoir and the user's breathing mask. The buffering reservoir in the digitally controlled PSA system 130 stabilizes the airflow, but the actual gas stream the user inhales can vary depending on many factors, e.g., generator efficiency, switching valve timing, leaks, or even backflow from the user's exhalation. By positioning the oxygen sensor at this final stage, just before the mask, the device measures the true FiO₂ that reaches the user. Accordingly, the oxygen sensor 210 ensures that the oxygen concentration delivered is exactly what the AI-controlled protocol intends, rather than relying on theoretical generator output. If the oxygen level at the mask falls below or rises above set safety thresholds (e.g., below a hypoxia floor or above a hyperoxia ceiling), the system may also trigger emergency hyperoxic bursts or even shut down delivery. In addition, because the sensor measures actual flow dynamics at the end of the circuit, discrepancies between commanded and measured values reveal leaks, blockages, or disconnections. Further, as a critical part of the closed-loop control, data from the oxygen sensor 210 feeds directly into the computation unit, which uses it along with physiological sensors (SpO₂, HRV, CGM, blood pressure, perfusion index) to continuously adapt protocol intensity in real time. It should be noted that, under certain circumstances, an oxygen sensor might not be required in actual applications. Instead, FiO₂ levels may be determined through a variety of pressure sensors installed along the pathway without using an oxygen sensor.

The breathing interface 160 may provide a single patient interface (e.g., full-face mask, nasal mask, or nasal cannula) for both hypoxic and hyperoxic phases without component swaps. It couples to the outlet of the digitally controlled PSA system 130 and maintains a comfortable, low-leak seal while minimizing dead space and inspiratory resistance. An exhalation pathway (e.g., integrated vent or valve) may be further included to manage CO₂ washout, and an optional sampling port may be included to support end-tidal or line pressure sensing. In some embodiments, the breathing interface 160 may include quick-release fittings, multiple sizes and seal geometries for fit, and biocompatible/cleanable materials suitable for repeated use.

The sensor suite 110 may provide multi-modal physiological inputs for closed-loop IHHT. In some embodiments, the suite includes at least one of: (i) a blood oxygen saturation (SpO₂) sensor; (ii) an HRV source obtained from ECG or PPG; and (iii) a continuous glucose monitor. Additional optional channels may include, but are not limited to, respiration (e.g., flow/pressure or impedance), end-tidal CO₂, perfusion index, skin or gas temperature, ambient pressure and humidity, and user-context sensors such as a 3-axis accelerometer/gyroscope. Sensors may be integrated on-device, connected via wired ports, or paired wirelessly (e.g., through wireless ingress 104), which are not limited in the present disclosure.

A blood oxygen saturation sensor measures the percentage of hemoglobin in the blood that is saturated with oxygen, typically expressed as SpO₂. Implemented as a pulse oximeter, a SpO₂ ring-style oximeter, or other proper formats, the blood oxygen saturation sensor uses light-based technology, emitting red and infrared wavelengths through body tissue (e.g., a fingertip or earlobe) and detecting how much light is absorbed. Since oxygenated and deoxygenated hemoglobin absorb light differently, the sensor may calculate oxygen saturation by analyzing the absorption ratio. In the IHHT training, SpO₂ readings are essential for tracking the hypoxic and hyperoxic phases, ensuring safety, and guiding the adaptive control algorithms.

Heart-rate variability refers to the variation in the time interval between successive heartbeats, a key indicator of autonomic nervous system balance and stress resilience. HRV may be derived from two main sensor modalities. The first one is ECG, which measures the electrical activity of the heart via skin electrodes, providing precise detection of the R-R intervals (the time between successive R-waves in the cardiac cycle). ECG-derived HRV is considered the gold standard for accuracy. The second one is PPG, which uses optical sensors (which may be in wearables like smartwatches) to detect blood volume changes in the microvascular bed of tissue. While less precise than ECG, PPG-based HRV is more convenient and may be collected from devices like finger clips or wristbands. In the IHHT training, HRV may serve as a measure of the body's autonomic response to hypoxic stress, complementing SpO₂ data in the AI's feedback loop.

Blood pressure is the hemodynamic guardrail. Hypoxia can raise blood pressure via sympathetic activation and peripheral vasoconstriction, but the magnitude and tolerance vary widely by individual and comorbidities. Using absolute ceilings (e.g., clinician-defined stop criteria) and relative changes from a personal baseline lets a user prevent unsafe cardiovascular strain while still delivering an effective stimulus. If a user has continuous beat-to-beat BP, slow-trend monitoring can catch rising afterload or exaggerated pressor responses; with intermittent cuff readings, pre-/mid-/post-session checks still reveal problematic deltas. BP also helps discriminate benign tachycardia from risky responses: a modest heart-rate rise with stable pressures is very different from a large systolic blood pressure (SBP)/diastolic blood pressure (DBP) jump or a sudden drop, suggesting vasovagal features. When BP breaches limits, the controller should extend recovery, reduce the next hypoxic window, or end the session.

Perfusion index (PI) is both a signal-quality sentinel and a physiologic window into peripheral vasomotor tone. Computed from the photoplethysmogram as the pulsatile-to-nonpulsatile ratio, PI plummets with cold extremities, sensor misplacement, or motion, exactly the conditions that make SpO₂ numbers untrustworthy. Using PI to gate decisions prevents the controller from chasing artifacts: if PI is below a quality threshold or drops abruptly, a user can pause closed-loop adjustments, prompt a sensor check, or switch sites. When PI is high-quality and stable, its physiologic changes add context: a sustained PI drop during hypoxia (with good signal) indicates sympathetic-driven vasoconstriction and rising autonomic load, supporting HRV-based down-titration even if SpO₂ looks acceptable. In short, PI ensures a user acts on the clean data first, and then doubles as a sensitive indicator of peripheral stress once the data are clean.

Furthermore, a continuous glucose monitor tracks glucose levels in interstitial fluid in near real-time, e.g., using a minimally invasive sensor inserted just under the skin. The sensor may measure glucose concentrations via enzymatic electrochemical reactions, and a transmitter sends this data wirelessly to a receiver or mobile device. CGMs provide glucose readings every few minutes, revealing trends and fluctuations throughout the day. In an IHHT setting, CGM data may add a metabolic dimension to protocol control, showing how the body responds to hypoxic stress in terms of glucose mobilization and clearance, and enabling a “neuro-metabolic guided” training approach, which is a control methodology that adapts therapy parameters in real time based on both autonomic nervous system signals (neuro) and metabolic state indicators (metabolic).

Wireless ingress 220 in the IHHT device refers to its ability to receive physiological data from the sensor suite 110 via wireless communication protocols such as Bluetooth or ANT+. This feature allows wearable sensors, like SpO₂ monitors, HRV trackers, and continuous glucose and blood pressure monitors, to transmit their readings directly to the computation unit 120 (which may be a part of the controller or coupled to the controller 150) without requiring physical cables. By eliminating wired connections, wireless ingress 220 enhances user comfort, supports unrestricted movement during training, and simplifies setup, making the device more suitable for both home and clinical environments. In some embodiments, the wireless ingress 220 receives the physiological data directly from the external sensors. In other embodiments, the wireless ingress 220 may receive the physiological data from a mobile device 230 associated with the user, where the mobile device 230 may include a mobile application 240 configured for monitoring signal collection and wireless transmission to the IHHT device. In some embodiments, the same or different application 240 on the mobile phone may further enable the user to select a training protocol, view and monitor the training session, modify or adjust protocol settings, view real-time data during the training session, review reports and/or analysis after the training session, etc.

Beyond convenience, wireless ingress 220 is critical for the system's real-time adaptive capabilities. In a neurovascularly guided and/or neuro-metabolically guided training approach, the AI-powered computation unit 120 may process multiple streams of physiological data with minimal delay. Wireless protocols like ANT+ are optimized for low-latency transmission of biometric data, ensuring that information such as SpO₂, HRV, and CGM, blood pressure readings arrive quickly enough for the AI to make immediate protocol adjustments. This capability is vital for maintaining safety, for instance, triggering an emergency hyperoxic burst if a user's oxygen saturation or metabolic markers cross unsafe thresholds.

Wireless ingress 220 also enables interoperability and scalability. Because it uses industry-standard protocols, the device may pair with a wide range of commercially available wearables from different manufacturers. This not only future-proofs the system against evolving sensor technology but also allows for flexible integration into broader health ecosystems, including mobile apps, remote monitoring platforms, and cloud-based analytics. Furthermore, wireless connectivity facilitates secure data transfer to external devices for session logging, progress tracking, and clinician review, extending the system's utility beyond the training session itself.

Controller 150 serves as the central operational unit that manages the delivery of oxygen streams to the user in accordance with the training protocol. It acts as the intermediary between the AI-powered computation unit 120 and the oxygen generation hardware (e.g., the digitally controlled PSA system 130), executing precise adjustments to oxygen concentration, interval timing, and gas flow direction. Based on real-time inputs, such as SpO₂, HRV, CGM, and blood pressure readings, as well as the reading from the oxygen sensor 210, the controller 150 may rapidly switch between hypoxic and hyperoxic gas streams, adjust flow rates, or initiate emergency safety responses like a high-SpO₂ burst to stabilize the user. This real-time responsiveness ensures both optimal training effectiveness and user safety throughout each session.

In the disclosed IHHT device 100, the controller 150 may be implemented as an embedded system within the device chassis 105, integrating both hardware and software components. On the hardware side, the controller 150 may interface with actuators such as solenoid valves, flow regulators, and sensors embedded in the PSA system 130. On the software side, the controller 150 may run firmware capable of interpreting commands from the AI computation unit 120, translating high-level protocol adjustments into precise mechanical actions. This design allows the controller 150 to handle both routine session management, such as interval transitions, and urgent, high-priority commands triggered by abnormal physiological data.

It should be noted that, in the IHHT device 100, the controller 150 may be configured for closed-loop operation, meaning it continuously receives live sensor feedback and modifies the output in real time. This makes it a key enabler of the neurovascularly guided and/or training approach, where protocol parameters are dynamically tuned based on the user's combined autonomic vascular and/or metabolic state. In addition, the controller 150 may store protocol presets, execute fail-safe routines in the event of sensor dropout, and manage wireless communications with peripheral devices, further expanding its role as the operational heart of the IHHT device.

Optionally, remote and cloud monitoring 250 may be provided for the IHHT device 100 to enable real-time and historical session data to be securely transmitted from the device to external platforms, such as clinician dashboards, mobile applications, or cloud-based analytics services. This capability may allow healthcare providers, coaches, or researchers to observe a user's training performance and physiological responses without being physically present. Key metrics like SpO₂ trends, HRV patterns, CGM, blood pressure readings, and protocol adjustments may be streamed live or synced after a session, enabling timely feedback, safety oversight, and long-term progress tracking.

From a technical standpoint, remote/cloud monitoring 250 relies on the device's wireless ingress 220 and outbound connectivity. Physiological data captured via Bluetooth or ANT+from wearable sensors is processed by the computation unit 120, then securely encrypted and transmitted to a cloud server via Wi-Fi, LTE, or another internet connection. Cloud infrastructure stores this information in structured formats, making it accessible for data visualization, AI-driven trend analysis, and integration with electronic health records (EHRs). This architecture also supports asynchronous review, so multiple stakeholders, such as physicians, physiologists, or athletic trainers, can access the same dataset from different locations.

In some embodiments, beyond oversight, cloud/remote monitoring 250 may enhance the adaptiveness and personalization of IHHT protocols. With aggregated historical data, the AI mode included in the computation unit 120 may identify long-term trends in a user's neurovascular and/or neuro-metabolic responses, enabling protocol refinements across weeks or months rather than just within a single session. In addition, cloud-based systems may issue proactive alerts, such as recommending a training pause if a user's physiological patterns suggest excessive strain, providing another layer of safety. This approach not only supports individualized care but also facilitates large-scale research studies by pooling anonymized data across many users to uncover broader insights into hypoxia-hyperoxia training outcomes.

The computation unit 120 in the IHHT device may function as the device's “AI brain”, integrating hardware, firmware, and software components to receive, process, and act upon real-time physiological data. Its primary role is to execute the adaptive control logic that enables neurovascularly and/or neuro-metabolically guided training, continuously analyzing incoming sensor data such as SpO₂, HRV, CGM, and blood pressure values and perfusion index to determine the optimal oxygen concentration, interval timing, and training intensity for the user.

Physically, the computation unit 120 may include one or more processors (CPUs or microcontrollers) connected to system memory, persistent storage, and I/O interfaces. These processors execute the AI algorithms, manage communication with peripheral devices, and control the timing and switching of the hypoxic and hyperoxic gas streams. Wireless interfaces such as Bluetooth, ANT+, or Wi-Fi may be integrated into the unit (or coupled to the unit) for sensor data ingestion and cloud connectivity. In some embodiments, the computation unit 120 may also feature a dedicated neural processing engine or GPU for accelerating machine learning model inference in real time.

In some embodiments, the computation unit 120 may run embedded firmware and higher-level software modules responsible for multi-modal sensor fusion, combining data from different physiological sources into a unified, real-time physiological stress score. This score reflects both the autonomic nervous system's state (via HRV and SpO₂) and the vascular and metabolic state (via CGM and/or blood pressure), enabling a closed-loop feedback system.

In some embodiments, the IHHT device 100 operates as a thin-client actuator. That is, the computation unit 120 on the device is not responsible for storing data, or any other type of logic. This data is stored in the cloud, as it is received through the mobile device 230, such as a smartphone, and the protocols and instructions are sent from the mobile device 230 or cloud. The computation unit 120 on the device is simply responsible for following the instructions sent from the mobile device 230 or cloud. For example, the mobile application 240 may acquire physiological data and relay it to a cloud service that computes the session protocol in real time, target FiO₂ trajectories, interval timings, rescue triggers, and safety thresholds. The cloud returns time-stamped, authenticated control instructions to the mobile device 230, which forwards them over a secure short-range link to the IHHT device 100. The computation unit 120 may then parse these messages to drive valves, compressors, and flow controllers accordingly; it does not persist user data or host protocol-selection logic, retaining only transient buffers needed to execute current commands.

In some embodiments, machine learning models, such as neural networks, decision trees, or regression models, may be included in the computation unit 120 or in the mobile device 230 or cloud. The model is mainly used to predict the user's optimal adaptive zone and dynamically adjust the training protocol. In some embodiments, the model may be pre-trained on general population data, then refined with an individual's historical data to create a personalized profile over time.

The exemplary machine model may be any one of a regression model (e.g., linear regression, logistic regression, or polynomial regression), decision tree, random forest, support vector machine, Naïve Bayes model, k-means cluster, or neural network (e.g., feed-forward networks, multilayer perceptron networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In some embodiments, the machine learning model includes a dimensionality reduction component for visualizing data, the dimensionality reduction component comprising any of a principal component analysis (PCA) component, or a T-distributed Stochastic Neighbor Embedding (t-SNE). In some embodiments, the machine learning model includes a neural network or a random forest, which is not limited in the present disclosure.

In some embodiments, the machine learning model includes a large language model (LLM), a generative AI, a combination thereof, or any derivative model thereof. In some embodiments, the LLM or generative AI model or another different model may keep communications with a user under the training besides the adaptive adjustment of the training process described above. For example, based on the sensor data, the LLM or generative AI model may consistently communicate with a user under the training to guide the user to control and adjust the breath (e.g., take deeper breaths at some timepoints) and the tempo (take different stride lengths at different timepoints) during the training process.

In some embodiments, the machine learning model includes one or more parameters, such as hyperparameters and/or model parameters. Hyperparameters are generally established prior to training. Examples of hyperparameters include the learning rate, depth or leaves of a decision tree, number of hidden layers in a deep neural network, number of clusters in a k-means cluster, a penalty in a regression model, and a regularization parameter associated with a cost function. Model parameters are generally adjusted during training. Examples of model parameters include weights associated with nodes in layers of the neural network, variables and thresholds for splitting nodes in a random forest, support vectors in a support vector machine, and coefficients in a regression model. The model parameters of the machine learning model are trained (e.g., adjusted) using the training data to improve the power of the machine learning model.

In some embodiments, the training data used for the training of the machine learning model(s) may include historical training and/or testing data obtained from a lot of users with different health conditions. In some embodiments, more than one machine learning model may be included in the disclosed adaptive training system. For example, at least one machine learning model may be used to determine a training protocol, and at least another machine learning model may be used to dynamically adjust a training protocol being used by a user.

In some embodiments, the computation unit 120 or the AI model included therein may compute a unified neurovascular and/or neuro-metabolic stress score for a user during a training session, and thus acquires a plurality of physiological signals in real time as described above. These signals may be preprocessed by applying digital filtering to remove noise and motion artifacts, normalizing each signal relative to a baseline established from historical user data, and optionally applying temporal smoothing (e.g., moving average or exponential smoothing) to reduce short-term variability while preserving trend data. In some embodiments, the computation unit 120 may further include certain components or modules that extract a plurality of features from each preprocessed signal. For example, from SpO₂ data, the components or modules may extract absolute oxygen saturation, deviation from baseline, and rate of change; from HRV data, the components or modules may extract time-domain metrics (e.g., root mean square of successive differences (RMSSD), standard deviation of NN intervals (SDNN)), frequency-domain metrics (e.g., low frequency/high frequency (LF/HF) ratio), and short-term variability; and from CGM data, the components or modules may extract instantaneous glucose level, magnitude of excursion from baseline, and recovery slope following a hypoxic event and from blood pressure data, the components or modules may discriminate benign tachycardia from risky responses.

In some embodiments, the extracted features may be normalized to a common numerical range and assigned weighting factors based on predetermined rules, clinical relevance, or personalization parameters derived from prior user sessions. In some embodiments, oxygenation features may be weighted more heavily when hypoxic thresholds are approached, HRV features may be weighted more heavily for early detection of autonomic stress, and CGM and/or blood pressure features may be weighted more heavily for detection of metabolic strain and recovery rate.

In some embodiments, the normalized and weighted features may be fused into the unified neurovascular and/or neuro-metabolic stress score using a fusion algorithm, which may include: (i) a weighted sum calculation; (ii) a rule-based decision tree; (iii) a statistical model; or (iv) a machine learning model such as a random forest, gradient boosting machine, or neural network trained on labeled datasets where stress state is known.

In some embodiments, the unified neurovascular and/or neuro-metabolic stress score may be updated at a fixed sampling interval (e.g., between one second and five seconds) and is compared to adaptive thresholds to determine a training action or may be updated in real-time when there is any detected change (over a predefined threshold) of physiological data for dynamic adjustment of an ongoing training session. If the score is within a first threshold range, the current training protocol is maintained; if the score is within a second, higher threshold range, the controller 150 may reduce hypoxic intensity or increase recovery interval duration; and if the score exceeds a third threshold range, the controller 150 may initiate an emergency hyperoxic burst.

In some embodiments, when the computation unit 120 determines an adjustment is needed, the computation unit 120 may communicate with the controller 150 to execute mechanical actions, such as opening or closing valves, switching between hypoxic and hyperoxic streams, or initiating an emergency hyperoxic burst. The computation unit 120 may also push protocol updates to the device's local display or mobile application, ensuring that the user or remote coach is informed of changes in real time.

Additionally or alternatively, the computation unit 120 may further compute a metabolic flexibility biomarker (“MFB”) from a user's CGM time-series in response to a standardized hypoxic step. In some embodiments, the biomarker may be computed during a standardized hypoxic challenge (e.g., defined FiO₂ and duration) delivered when motion is low and sensor quality flags are adequate. In some embodiments, the computation unit 120 may map raw features to a unitless MFB score (e.g., 0-1, where higher=better flexibility) using a weighted combination.

In some embodiments, the computation unit 120 may consult the biomarker-linked guardrails while dosing for hypoxia. If excursion exceeds a user-specific limit or recovery speed falls below a threshold, the IHHT device 100 may automatically (i) raise the minimum FiO₂, (ii) shorten the current or next hypoxic interval, (iii) extend recovery, and/or (iv) trigger a brief hyperoxic rescue burst. Progression to the next step is withheld until a recovery is met. Rate limiters and hysteresis may prevent oscillation.

In some embodiments, the computation unit 120 may maintain a trend of MFB and adjust starting parameters for subsequent sessions: improved MFB for recent sessions permits small, bounded dose escalations (e.g., +≤10% hypoxic duration, −≤2 pp FiO₂), whereas deteriorating MFB tightens guardrails (shorter hypoxia, longer recovery, higher floor FiO₂). All adjustments may remain within clinician-approved bounds. In addition, certain hard safety limits may not be relaxed.

In some embodiments, if CGM is unavailable or low-confidence, the computation unit 120 may fall back to surrogate biomarkers (e.g., HRV recovery and/or FiO₂), apply conservative defaults, and flag the session. A clinician portal may review biomarker histories, set allowable ranges, and lock minimum thresholds. All computations, limits, and overrides may be versioned and auditable.

In some embodiments, the computation unit 120 may include or be coupled to storage 260 for both transient and long-term data. Short-term buffers may be used for high-speed processing during a session, while non-volatile memory may store historical training records, AI model parameters, and firmware. Security protocols, such as encrypted data transmission and secure boot, may be used to protect user information, particularly when transmitting data to remote/cloud monitoring systems.

In essence, the computation unit 120 transforms the IHHT device 100 from a static oxygen generator into an intelligent, adaptive training system, capable of understanding a user's unique neurovascular and/or neuro-metabolic signature, making split-second adjustments, and learning over time to deliver safer, more effective, and more personalized training outcomes.

While not shown, in some embodiments, the IHHT device 100 further includes certain safety monitors. In some embodiments, these safety monitors may supervise delivery independent of the control model. The monitors may enforce safety thresholds such as a minimum SpO₂ floor, cap hypoxic exposure within a rolling window, and detect leaks/occlusions by comparing commanded versus measured flow and/or pressure. On violation of one or more of these safety thresholds, the IHHT device 100 may transition to a protective state (e.g., hyperoxic or ambient supply), alert the user, and record a timestamped fault event. Watchdog timers may verify sensor freshness and valve response, and monitored thresholds and actions may be clinician-configurable within prescribed limits.

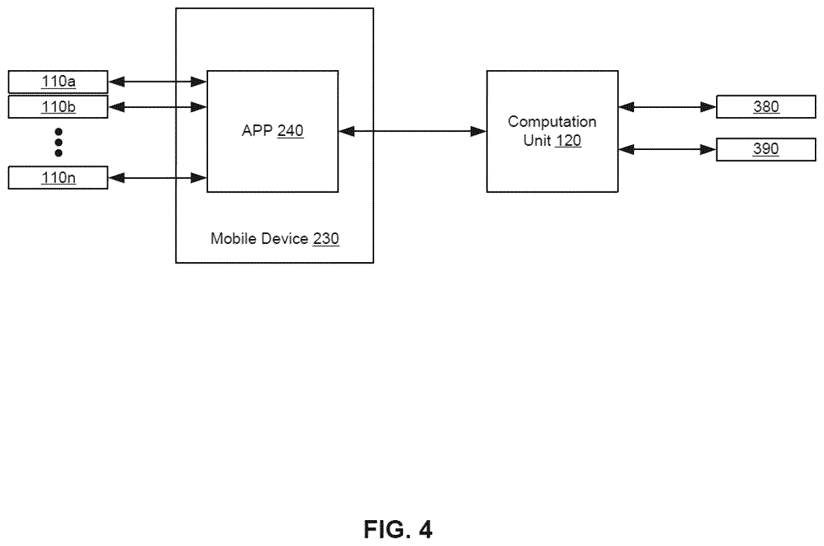

In the following, some specific hardware components, such as the digitally controlled PSA system 130 and buffering reservoir 140, are further described in detail. Specifically, FIG. 3 illustrates an example digitally controlled PSA system 300, according to some embodiments, and FIG. 4 illustrates an example buffering reservoir 400, according to some embodiments.

### Example of Digitally Controlled PSA System

As described earlier, in some embodiments, the IHHT device 100 includes a digitally controlled PSA system 130 configured to selectively generate a hypoxic gas stream and a hyperoxic gas stream within a single integrated system. PSA systems may include molecular sieves as the medium where gas molecules adhere and detach based on controlled pressure variations, which is a gas separation process that utilizes the differing affinities of gas molecules for an adsorbent material under varying pressure. Essentially, it works by selectively adsorbing (attaching to a surface) certain gases at higher pressures and then releasing them at lower pressures. This cyclic process allows for the separation and purification of gases like hydrogen, nitrogen, and oxygen, which can then be used to generate a hyperoxic gas stream and a hypoxic gas stream.

As illustrated in FIG. 3, the digitally controlled PSA system 130 may include a couple of adsorption towers 350a and 350b to produce both a nitrogen-enriched, oxygen-depleted gas stream and an oxygen-enriched gas stream having a fractional inspired oxygen concentration (FiO₂) exceeding 80%. Briefly, when the adsorption tower 350a is in the adsorption stage, zeolite may preferentially capture nitrogen, so the product leaving the adsorption tower 350a is an oxygen-rich hyperoxic gas stream. At the same time, while the adsorption tower 350a is in the adsorption stage, the adsorption tower 350b is in the desorption stage or regeneration stage, where it is depressurized to desorb the trapped nitrogen, so as to generate a nitrogen-rich hypoxic gas stream. The two adsorption towers then swap roles every few seconds, so that the hypoxic and hyperoxic gas streams may be continuously provided by the alternated outputs of the two gas streams.

It should be noted that, while in the above example, the molecular sieve media is selected to preferentially adsorb nitrogen molecules, the present disclosure is not limited thereto. In some embodiments, a molecular sieve media is selected to preferentially adsorb oxygen molecules, thereby allowing nitrogen-rich gas stream to be desorbed while the oxygen-rich gas stream is adsorbed.

In some embodiments, ambient air enters the IHHT device 100 through two separate inlet points, each feeding a dedicated filter 310a or 310b. These filters 310a/310b remove particulate matter and contaminants, ensuring that only clean air reaches the compressor 320. Once filtered, the air is drawn into the compressor 320, where its pressure is increased to the operational level required for PSA separation. The high-pressure air then passes through a heat exchanger 330, which may condition the air to a proper temperature, preventing excess heat or cold from affecting the adsorption process. From the heat exchanger 330, the conditioned compressed air flows into a central control valve assembly 340.

The central control valve assembly 340 acts as the traffic director of the system, determining whether the incoming compressed air is routed into absorption tower one (tower 350a) or absorption tower two (tower 350b). As described earlier, each tower 350a or 350b contains zeolite molecular sieve material that selectively adsorbs nitrogen from the incoming air, leaving an oxygen-rich gas mixture, which is then purged from one of the towers 350a or 350b and vented through dedicated equilibrium canister 360a or 360b. An equilibrium canister is a small, rigid, oxygen-clean plenum installed on the hyperoxic PSA product line. It equalizes (1) pressure ripple and (2) O₂ concentration ripple coming out of the PSA towers, so the downstream selector valve always sees a stable, high-purity (e.g., >80% FiO₂) reservoir. It also provides short “surge” capacity for brief rescue bursts without purity sag. The outlet of the equilibrium canisters 360a and 360b is coupled to a selector valve 365 that alternately directs the hyperoxic gas stream to a buffering reservoir 140a or optionally to the central control valve assembly 340, which can be then re-routed to an adsorption tower 350a or 350b that is generating the oxygen-rich gas stream, so that the hyperoxic gas stream can be reused to improve the efficiency of the PSA system 130.

While one tower is actively producing hyperoxic air, the other is in a regeneration cycle. During regeneration, nitrogen-rich exhaust is purged from the tower and vented through dedicated nitrogen mufflers 370a or 370b, which are engineered to reduce cyclic PSA/valve noise and breath-path hiss without adding noticeable resistance or shedding fibers into the patient circuit. The mufflers 370a, 370b may include a reactive stage having one or more Helmholtz resonators tuned to attenuate cyclic pressure and concentration ripple from a pressure-swing adsorption cycle, and an absorptive stage including a micro-perforated panel disposed within an expansion chamber to attenuate broadband valve noise. The outlets of the mufflers is coupled to a selector valve 375 that alternately directs the hypoxic gas stream to a buffering reservoir 140b or optionally to the central control valve assembly 340, which can be then re-routed to a adsorption tower 350a or 350b that is generating the nitrogen-rich gas stream, so that the hypoxic gas stream can be reused to further improve the efficiency of the PSA system 130. In some embodiments, the routing of the selector valve 375, as well as the selector valve 365 mentioned earlier, can be automatically controlled by the computation unit 120 and/or further manually adjusted under certain circumstances, which are not limited in the present disclosure.

After the routing through the selector valve 365 or 375, each hyperoxic or hypoxic gas stream may be guided into a respective buffering reservoir 140a or 140b, both of which are internal reservoirs within the chassis 105. The buffering reservoir 140a/140b may include an internal, chassis-integrated reservoir that acts as a pneumatic low-pass filter between the PSA system 130 and the breathing interface 160. It smooths flow and pressure transients during phase changes and inspiratory peaks, shortens the effective pneumatic path versus external bags and long hoses, and thereby improves control responsiveness and user comfort. In some embodiments, the buffering reservoir 140a, 140b may include embedded pressure (and optional flow/temperature) sensing for closed-loop supervision, a pressure-relief pathway to prevent over-pressurization, and, optionally, certain service features such as an antimicrobial liner and accessible hatch. The integrated placement reduces external leak points, footprint, and setup steps while providing stable delivery of the commanded FiO₂. It should be noted that, while two buffering reservoirs are included in the disclosed digitally controlled PSA system 130, in some embodiments, only one buffering reservoir is included. For example, in some embodiments, there is only one buffering reservoir for the hypoxic gas stream, but there is no buffering reservoir for the hyperoxic gas stream.

The switching valve 380 is a computer-controlled manifold that (i) selects between the hypoxic and hyperoxic gas streams from the buffering reservoir 140a or 140b, and (ii) in some operation modes proportionally blends a selected stream with ambient air to achieve a commanded FiO₂ before delivery to a patient output port 390 coupled to the breathing interface 160. It is specified for fast actuation (e.g., sub-100 ms), low dead volume, and low leakage, and may include position feedback and downstream flow/pressure sensing (e.g., flow sensor 210) for closed-loop setpoint control. One-way isolation may be used to prevent cross-flow between paths. In some embodiments, certain firmware may be used to apply hysteresis and rate limits to avoid oscillation during frequent transitions. In addition, a fail-safe bias to a breathable state (e.g., ambient or hyperoxic) may be further imposed on detected fault or power loss. Calibration routines verify valve linearity and blend accuracy over time.

In some embodiments, the switching valve 380 may be further used as a mixing valve, allowing a mixing of both hypoxic and hyperoxic gas streams supplied by the buffering reservoirs 140a and 140b. For example, for timely adjustment, a brief mixture of hypoxic gas stream into a main hyperoxic gas stream may allow the delivered gas mixture to adjust a little without necessarily switching between two dedicated gas streams.

While not shown in FIG. 3, in some embodiments, to manage continuous gas production during periods of low or paused user demand, the digitally controlled PSA system 130 further includes one or more pressure relief mechanisms. In one example, an internal buffering reservoir 140 may be fluidly coupled to a first pressure relief valve configured to vent excess gas to the ambient atmosphere when a pressure within the reservoir exceeds a predetermined threshold. In some embodiments, a gas output pathway is fluidly coupled to a second pressure relief valve configured to vent excess gas to the ambient atmosphere when a pressure within the pathway exceeds a predetermined threshold. The provision of these pressure relief valves maintains safe and stable operation of the digitally controlled PSA system 130 and prevents over-pressurization of system components.

In the disclosed digitally controlled PSA system 130, the architecture overcomes the “two-mask restriction” inherent in other existing PSA-based IHHT systems and, for the first time, enables full digital control of hypoxic and hyperoxic delivery through a single user interface. In an example embodiment illustrated in FIG. 4, a computation unit 120 is configured to receive commands and protocol instructions from a user's mobile device 230 via a wireless communication link (e.g., Bluetooth). The mobile device application 240 is configured to execute personalized training protocols that adapt in real time based on physiological data received from one or more wearable sensors 110a, 110b, . . . , 110n, including, for example, sensors for blood oxygen saturation (SpO₂), heart-rate variability (HRV), continuous glucose monitoring, blood pressure, and perfusion index (which may be directly sent to the computation unit 120 or through the mobile device 230). The application 240 is further configured to transmit control commands to the computation unit 120 (and/or controller 150 included therein) to adjust hypoxic intensity, flow rate, or to operate a switching valve 380 to deliver brief, intra-session hyperoxic bursts for safety purposes and/or protocol optimization.

In some embodiments, adjustment of hypoxic intensity is achieved by the computation unit 120 transmitting digital control signals to the digitally controlled PSA system 130 to modify one or more operational parameters, such as cycle time and airflow rate. To ensure precision, an oxygen sensor 210 is disposed within a fluid pathway leading to a single user outlet (e.g., after the switching valve 380). The computation unit 120 is configured to receive real-time oxygen concentration data from the oxygen sensor 210, compare the received concentration to a target concentration specified by the active protocol, and execute fine-tuning adjustments to the operational parameters of the digitally controlled PSA system 130 in a closed-loop manner to maintain the target concentration.

### Example of Buffer Reservoir

In the following description, an example buffering reservoir 140 is further described by taking an expandable buffer bag having an accordion-style geometry as an example. However, it is to be noted that buffering reservoir 140 may take other forms. For example, in some embodiments, the buffering reservoir 140 may be a rigid cylinder under certain configurations.

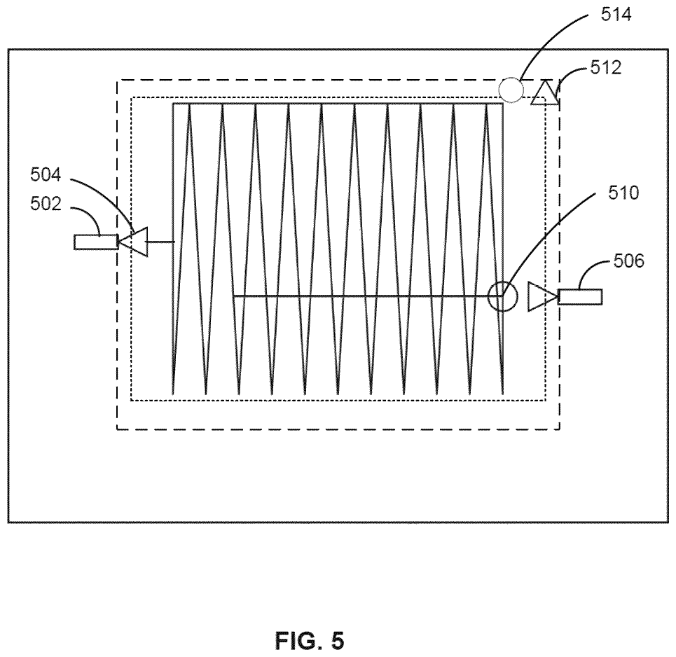

In the illustrated embodiment in FIG. 5, the expandable body may be formed from a flexible, gas-impermeable material configured to withstand repeated expansion and contraction cycles without material fatigue, deformation, or leakage. The accordion-style geometry is configured to permit controlled volumetric expansion to accommodate incoming flow surges and controlled contraction to maintain downstream output flow during periods of reduced upstream supply.

In some embodiments, the buffering reservoir 140 includes an inlet port 502 in fluid communication with an output of the equilibrium canister 360 or directly with an adsorption tower 350. In some embodiments, the integrated buffering reservoir 140 may further include an inlet check valve 504 positioned between the output of the equilibrium canister 360 or the adsorption tower 350. The inlet check valve 504 is configured to permit gas flow into the buffering reservoir 140 while preventing reverse flow toward the upstream generation components. This unidirectional control maintains separation between hypoxic and hyperoxic gas streams and prevents pressure backflow from the buffering reservoir 140 into the equilibrium canister 360 or the adsorption tower 350 during pressure switching or compressor idle cycles.

An outlet port 506 of the buffering reservoir is fluidly coupled to the switching valve 380 that is digitally controlled. The switching valve 380 may allow gas flow to exit the buffering reservoir toward the delivery interface while preventing ingress of ambient air or exhaled breath back into the reservoir. This maintains gas purity and protects internal components from contamination or moisture intrusion during user inhalation and exhalation cycles. The buffering reservoir 140 is thereby operable to smooth flow transitions resulting from switching events, compressor cycling, or blending ratio adjustments.

In certain embodiments, a flow sensor 510 may be disposed at or near the outlet port 506 of the buffering reservoir and is configured to measure the volumetric flow rate of the gas delivered to the switching valve 380 and further to the breathing interface. Data from the flow sensor 510 may be communicated to the computation unit 120 or controller 150 to monitor user breathing patterns, verify protocol adherence, and adjust oxygen delivery parameters in real time.

In some embodiments, the buffering reservoir 140 may further incorporate a relief valve 512 configured to vent excess gas pressure if the internal reservoir pressure exceeds a predetermined threshold. The relief valve 512 may be spring-loaded or otherwise biased to open automatically under overpressure conditions, thereby preventing damage to the buffering reservoir, downstream components, or the breathing interface. In some embodiments, the relief valve 512 is positioned and oriented to vent gas safely away from the user and sensitive electronics. In some embodiments, the buffering reservoir 140 may additionally include a pressor sensor 514 to monitor the pressure before the release.

In the disclosed IHHT device 100, the integration of the buffering reservoir 140 within the chassis 105 is configured to reduce device footprint, improve portability, and enhance durability by physically shielding the buffering reservoir from accidental puncture, kinking, or environmental damage. The internal placement also eliminates the need for external, inflatable bag-type reservoirs commonly used in other existing IHHT devices, thereby simplifying user setup and reducing potential points of failure.

In some embodiments, the buffering reservoir 140 is operatively controlled in coordination with the computation unit 120 or controller 150, such that gas flow characteristics within the reservoir may be dynamically adjusted to meet real-time protocol requirements determined from physiological data inputs, including but not limited to SpO₂, HRV, CGM and blood pressure data.

### Example Implementation

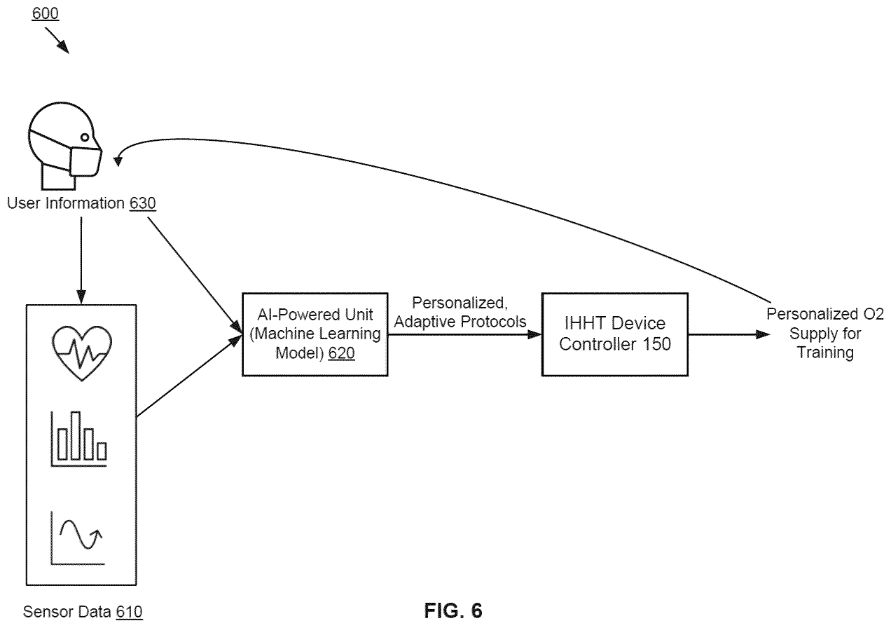

Referring now to FIG. 6, an example method 600 for operating the AI-powered IHHT device 100 is described, in accordance with an embodiment. Briefly, user information 630 may be first collected. For example, a person may log into the computation system of the device (e.g., through the mobile application 240 of the mobile device 230). Alternatively, if the device is dedicated to personal use, the person may be recognized automatically by default. Once the user information 630 is collected, it is provided, together with sensor data 610 obtained from one or more physiological sensors (e.g., sensor suite 110), to an AI-powered unit (machine learning model) 620.

The AI-powered unit 620 is configured to identify a personalized, adaptive training protocol for the user. The identified protocol may be the same as a previously used protocol for that user or a new protocol derived from population-level data for users with similar personal information, such as age, weight, or health status. In some embodiments, the computation unit 120 of the device 100 may also allow the user to select a training protocol from a set of predefined protocols. Once the protocol is determined, it is transmitted from the AI-powered unit 620 to the IHHT device controller 150, which manages the oxygen generation system to deliver a personalized O₂ supply for training according to the specified adaptive protocol.

During the training session, the user may be monitored continuously through one or more wearable or non-wearable sensors, generating sensor data 610 in real time. This sensor data may include parameters such as SpO₂, HRV, CGM, blood pressure, and other possible physiological metrics relevant to training adaptation. The real-time data is sent back to the AI-powered unit 620, which evaluates whether adjustments to the current protocol are necessary. In one example, a unified neurovascular and/or neuro-metabolic stress score from SpO₂, HRV, CGM, and blood pressure data may be computed in the computation unit, which may be used to determine if an adjustment is necessary. If an adjustment is warranted, the AI-powered unit 620 generates an updated protocol and sends it to the IHHT device controller 150, which then alters the oxygen supply parameters accordingly. This closed-loop process may be repeated throughout the session, enabling continuous optimization of the training protocol in real time. In another embodiment, a metabolic flexibility biomarker may be similarly monitored through the sensed data for protocol adjustment.

In some embodiments, the AI-powered unit 620 may also generate personalized recommendations based on the analysis of past training sessions combined with user information 630. For example, older individuals may be advised to follow less intense training regimens, while younger individuals may be guided toward more demanding protocols. Additionally, based on historical and session-specific data, the AI-powered unit 620 may recommend complementary activities, such as targeted exercise routines or lifestyle adjustments, to further enhance the effectiveness of IHHT-based training.

### Personalization

In some embodiments, the computation unit 120 may maintain a per-user profile that is updated across sessions from time-aligned physiological inputs (e.g., SpO₂ trajectories, HRV suppression/recovery, and kinetics), device-side circuit measurements (e.g., flow/pressure dynamics, leak), and protocol metadata (e.g., hypoxic/hyperoxic durations, FiO₂ setpoints, rescue events). The computation unit may derive summary features per interval and per session (e.g., time-to-SpO₂-floor, maximum desaturation rate, HRV nadir and recovery half-time, glucose excursion area-under-curve and recovery slope) and update a vector of per-user response parameters using a bounded adaptive estimator (e.g., exponentially weighted moving average with outlier rejection and confidence weighting by data quality flags).

In some embodiments, the computation unit 120 may map learned parameters to individualized safety thresholds and control bounds, including but not limited to minimum SpO₂ floor, maximum permitted desaturation rate, maximum hypoxic exposure per rolling window, maximum allowed CGM excursion, and minimum required recovery slope before advancing dose. Personalization may tighten guardrails for sensitive users (e.g., lower SpO₂ floor, shorter hypoxic windows when glycemic sensitivity is high) and relax within clinician-approved limits when repeated sessions demonstrate improved tolerance.

For each new session, the computation unit 120 may select initial FiO₂ targets, interval durations, ramp rates, and rest periods by referencing the per-user parameters and recent biomarker trends. For example, when the hypoxic tolerance index and glycemic sensitivity both improve for N consecutive sessions, the next session may increase hypoxic duration by a bounded step (e.g., ≤10%) and/or lower target FiO₂ by a small decrement (e.g., ≤1-2 percentage points), subject to guardrails. Conversely, if CGM excursion exceeds a user-specific limit or HRV recovery half-time worsens beyond a threshold, the schedule may be automatically de-escalated (shorter hypoxic intervals, longer recovery, higher minimum FiO₂) and a hyperoxic rescue burst trigger is made more sensitive. During a session, the controller may adjust the schedule in real time from a neurovascular and/or neuro-metabolic stress score or biomarker score while the computation unit logs cause-and-effect tuples (state, action, response), as described above. Post-session, these tuples may update the per-user parameters and optionally a lightweight personalization layer so that future sessions start closer to the user's current capacity.

### System And/or Computer Embodiments

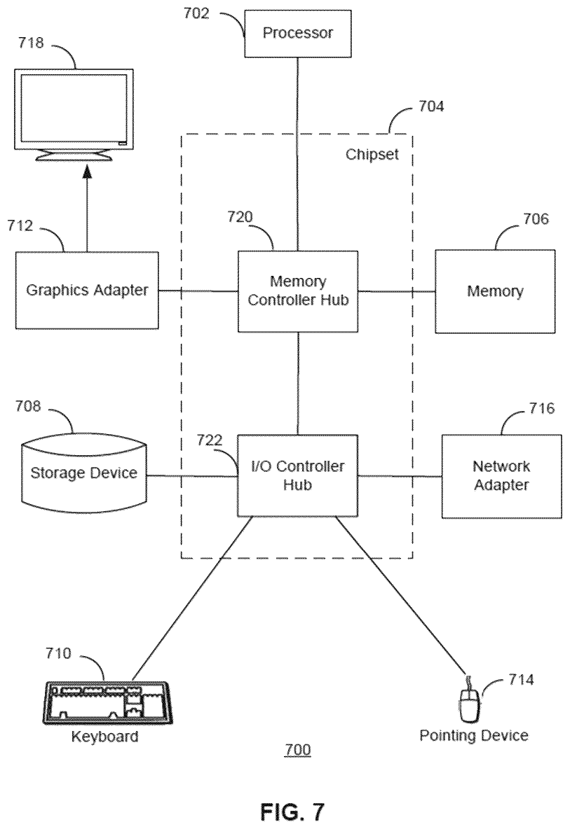

FIG. 7 depicts an example computing device 700 for implementing systems and methods described in reference to FIGS. 1-6. Examples of a computing device may include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, edge devices, IoT devices, and the like. In some embodiments, the computing device 700 may operate as an AI-powered unit. Thus, the computing device 700 may train and/or deploy machine learning models for monitoring IHHT training sessions.

In some embodiments, the computing device 700 includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input interface 714, and a network adapter 716 are coupled to the I/O controller hub 722. Other embodiments of the computing device 700 have different architectures.

The storage device 708 is a non-transitory computer-readable storage medium such as a hard drive, compact disk read-only memory (CD-ROM), DVD, or a solid-state memory device. The Memory 706 holds instructions and data used by the processor 702. The input interface 714 is a touch-screen interface, a mouse, a trackball, or other types of input interface, a keyboard, or some combination thereof, and is used to input data into the computing device 700. In some embodiments, the computing device 700 may be configured to receive input (e.g., commands) from the input interface 714 via gestures from the user. The graphics adapter 712 displays images and other information on the display 718. The network adapter 716 couples the computing device 700 to one or more computer networks.

The computing device 700 is adapted to execute computer program modules for providing the functionality described herein. As used herein, the term “module” refers to computer program logic used to provide the specified functionality. Thus, a module may be implemented in hardware, firmware, and/or software. In one embodiment, program modules are stored on the storage device 708, loaded into the memory 706, and executed by the processor 702.

The types of computing devices 700 may vary from the embodiments described herein. For example, the computing device 700 may lack some of the components described above, such as graphics adapters 712, input interface 714, and displays 718. In some embodiments, a computing device 700 may include a processor 702 for executing instructions stored in a memory 706.

### Example IHHT System

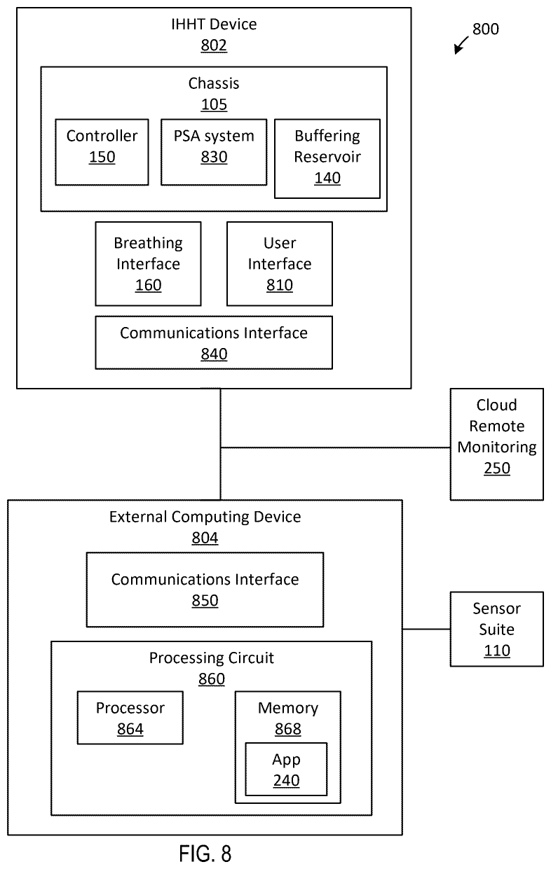

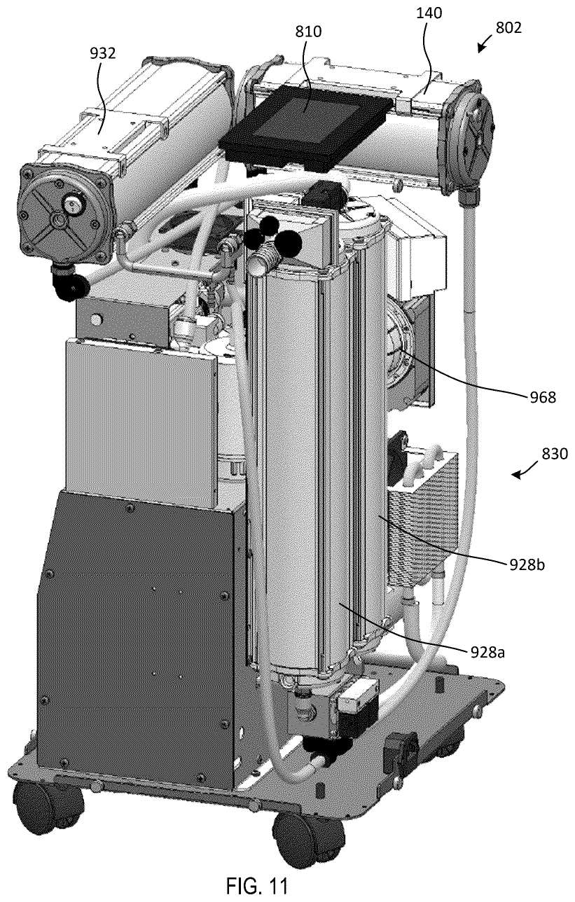

Referring generally to FIG. 8-18, systems and methods for controlling an IHHT device are shown. Referring to FIG. 8, a second example IHHT system, shown as system 800 is described. The system 800 may include an IHHT device 802 and an external computing device 804 (e.g., a mobile device, a patient's device, etc.). The IHHT device 802 may be coupled to the external computing device 804 and configured to receive instructions and/or data from the external computing device 804 to control operation of the IHHT device 802. The IHHT device 802 may be substantially similar to the IHHT device 100 described with respect to FIGS. 1-6 unless otherwise indicated to the contrary. Features of the IHHT device 100 described with respect to FIGS. 1-6 may be present in the IHHT device 802 described with respect to FIGS. 8-13 and features of the IHHT device 802 may be present in the IHHT device 100.

As described in greater detail herein, the IHHT device 802 differs from the IHHT device 100 in that the IHHT device 802 employs an asymmetric buffering topology in which the hypoxic gas stream is buffered through an internal gas buffer to smooth PSA cycling transients for safe patient delivery, while hyperoxic and normoxic gas streams bypass the internal buffer entirely through separate delivery paths. Furthermore, the IHHT device 802 includes a blower configured to provide ambient air and a software-synchronized activation of the blower during a PSA pressure equalization phase. Accordingly, the IHHT device 802 is a self-contained unit that receives ambient air, generates both hyperoxic and hypoxic gas streams via pressure swing adsorption (PSA), stores the hypoxic gas stream in an internal gas buffer, and delivers multiple gas states through a single patient breathing interface 160. The IHHT device 802 is designed for personal assignment to a single patient for home-based use. The IHHT device 802 operates as a thin-client receiving all treatment commands from the external computing device 804 via a wireless communication link. For example, the IHHT device 802 may not include the computation unit 120. Instead, the computation unit 120 and/or functions of the computation unit 120 may be included in the external computing device 804 to control operation of the IHHT device 802.

As shown in FIG. 8, the system 800 includes the sensor suite 110. The sensor suite 110 may be coupled to the external computing device 804 and configured to provide sensor data to the external computing device 804. The external computing device 804 may then control one or more operations of the IHHT device 802 as described herein.

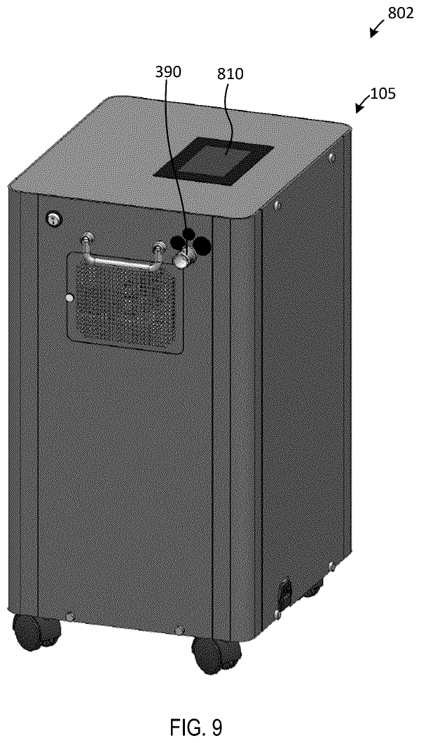

As shown in FIG. 8, the IHHT device 802 includes the chassis 105, the buffering reservoir 140, the controller 150, and the breathing interface 160. The chassis 105 may house and/or support the various components of the IHHT device 802. For example, the chassis 105 may house and/or support the buffering reservoir 140 and the controller 150. The breathing interface 160 may be coupled to the chassis 105 and extend outwards from the chassis 105. For example, and as shown in FIG. 9, the patient output port 390 may extend outwards from the chassis 105 and couple to the breathing interface 160 (e.g., a tube of the breathing interface 160). As described in greater detail herein, the controller 150 may be configured to receive control signals from the external computing device 804 to control operation of various components of the IHHT device 802

As shown in FIGS. 8 and 9, the IHHT device 802 further includes a user interface 810. The user interface 810 may be coupled to the chassis 105 (e.g., a top panel of the chassis 105) such that the user interface 810 is accessible to a user and/or an operator of the IHHT device 802. The user interface 810 may include one or more input devices (e.g., keyboards, displays, touchscreens, microphones, buttons, dials, switches, etc.) configured to receive input from the user and/or the operator. The user interface 810 may include one or more output devices (e.g., displays, speakers, etc.) configured to provide instructions to the user and/or the operator. The user interface 810 may be communicatively coupled to the controller 150 and configured to provide user commands to the controller 150 and receive control signals from the controller 150.

As shown in FIGS. 8 and 10-12, and described in greater detail herein, the IHHT device 802 further includes a gas delivery system, shown as PSA system 830. The PSA system 830 may be housed within the chassis 105 and may be configured to selectively generate a hypoxic gas stream, a hyperoxic gas stream, and/or a normoxic gas stream (e.g., an ambient air stream). The PSA system 830 may be substantially similar to the PSA system 130 and may include various components of the PSA system 130.

As shown in FIG. 8, the IHHT device 802 further includes a communications interface 840. The communications interface 840 may be a combination of hardware and software configured to facilitate wireless and/or wired communication between the IHHT device 802 and the external computing device 804. The communications interface 840 may include one or more transceivers, radios, antennas, and/or ports for communicating according to one or more communication protocols. By way of example, the communications interface 840 may support short-range wireless communication, including Bluetooth communication such as Bluetooth Classic and/or Bluetooth Low Energy (BLE), ANT+communication, local area network communication including Wi-Fi communication, cellular communication, near-field communication, serial communication, universal serial bus communication (USB), Ethernet communication, and/or other suitable wired and/or wireless communication techniques. The communications interface 840 may facilitate bidirectional communication such that data, measurements, instructions, commands, status information, alarms, and/or other information may be transmitted from the IHHT device 802 to the external computing device 804 and/or from the external computing device 804 to the IHHT device 802.

The communications interface 840 may be communicatively coupled to the controller 150. For example, the communications interface 840 may provide data and/or commands received from the external computing device 804 to the controller 150 and may transmit data from the controller 150 to the external computing device 804. In this way, the communications interface 840 may operate as an input/output pathway between the IHHT device 802 and the external computing device 804. The controller 150 may receive control signals, treatment instructions, operating parameters, timing information, software updates, calibration data, safety thresholds, and/or other information from the external computing device 804 (e.g. via the communications interface 840) to control operation of one or more components of the IHHT device 802. For example, the controller 150 may control one or more valves, compressors, blowers, fans, adsorption towers, alarms, and/or user interface components (e.g., the user interface 810) of the IHHT device 802 based on instructions received from the external computing device 804.

As shown in FIG. 8, the external computing device 804 may include a communications interface 850 and a processing circuit 860. The communications interface 850 may be configured to communicate with the communications interface 840 of the IHHT device 802 and to communicate with one or more components of the sensor suite 110. The communications interface 850 may include one or more transceivers, radios, antennas, and/or ports for communicating according to one or more communication protocols. By way of example, the communications interface 850 may support short-range wireless communication, including Bluetooth communication such as Bluetooth Classic and/or Bluetooth Low Energy (BLE), ANT+ communication, local area network communication including Wi-Fi communication, cellular communication, near-field communication, serial communication, USB communication, Ethernet communication, and/or other suitable wired and/or wireless communication techniques. The communications interface 850 may facilitate bidirectional communication such that physiological data, measurements, instructions, commands, status information, alarms, configuration information, and/or other information may be transmitted between the external computing device 804 and the IHHT device 802 and/or between the external computing device 804 and the sensor suite 110.

The processing circuit 860 may include a processor 864 and a memory 868. The processor 864 may be a general purpose or special purpose processor, an application specific integrated circuit (ASIC), one or more field programmable gate arrays (FPGAs), a group of processing components, or other suitable processing components. The processor 864 may be configured to execute computer code and/or instructions stored in the memory 868 or received from another computer readable medium or device.

The memory 868 may include one or more devices (e.g., memory units, memory devices, storage devices, etc.) for storing data and/or computer code for completing and/or facilitating the various processes described herein. The memory 868 may include random access memory (RAM), read only memory (ROM), hard drive storage, temporary storage, nonvolatile memory, flash memory, optical memory, or any other suitable memory for storing software objects and/or computer instructions. The memory 868 may include database components, object code components, script components, or any other type of information structure for supporting the various activities and information structures described herein. The memory 868 may store instructions that, when executed by the processor 864, cause the processor 864 to perform one or more operations described herein.

The memory 868 may store treatment protocols (e.g., hypoxic protocols, recovery protocols, hyperoxic protocol, normoxic protocol, etc.), protocol parameters (e.g., target concentrations, interval durations, threshold parameters etc.), decision rules, patient-specific parameters (e.g., user data, user profiles, etc.), calibration information, communication instructions, and/or software modules for processing sensor data and/or user data and generating control instructions for the IHHT device 802.

The sensor suite 110 may include one or more physiological sensors configured to generate physiological measurements associated with a user during an IHHT session. As described herein, the sensor suite 110 may include, for example, one or more SpO₂ sensors, HRV sensors, ECG sensors, PPG sensors, blood pressure sensors, CGM sensors, perfusion index sensors, and/or other physiological sensors. One or more sensors of the sensor suite 110 may transmit measurements (e.g., sensor data) to the external computing device 804 via the communications interface 850. The external computing device 804 may be configured to receive data and/or measurements from the sensor suite 110 and process the data and/or measurements to determine one or more control actions for the IHHT device 802.

In operation, the external computing device 804 (for example, a smartphone executing the treatment protocol application 240) receives physiological data from the sensor suite 110 and/or user data from a user (e.g., upon starting a session), processes the sensor data and/or the user data to determine protocol parameters including recovery interval composition, and transmits commanded parameters to the IHHT device 802 via Bluetooth Low Energy (BLE) or another short-range wireless protocol. The commanded parameters include at least FiO₂ target, mode selection, interval timing, and recovery interval composition (hyperoxic sub-interval duration and FiO₂, normoxic sub-interval duration). The controller 150 receives these commands and drives the PSA system 830 to achieve the commanded gas state at the patient output port 390.

### Example Gas Delivery System

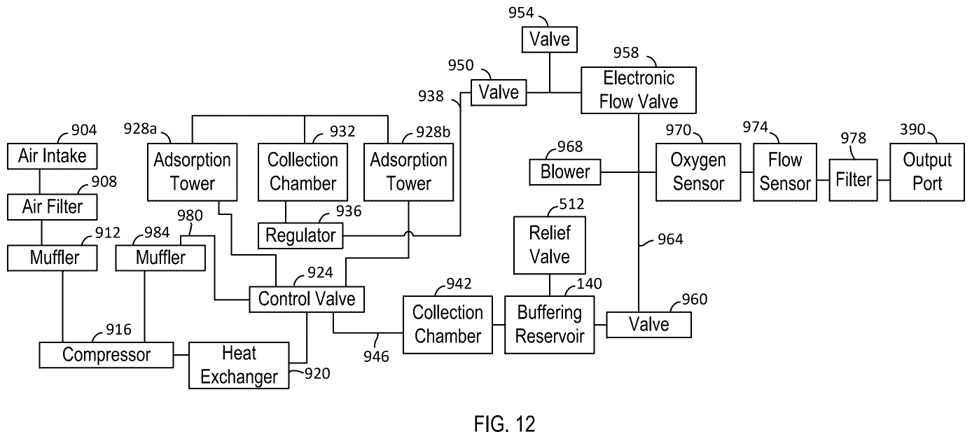

Referring now to FIG. 12, the IHHT device 802 and the PSA system 830 are shown in greater detail, according to an example embodiment. As shown in FIG. 12, the PSA system 830 includes an air intake 904 (e.g., an air vent, an air inlet, etc.). The air intake 904 is configured to receive ambient air surrounding the IHHT device 802 and provide the ambient air to additional components of the PSA system 830. The air intake 904 may be coupled to the main chassis 105. For example, the air intake 904 may include one or more openings, vents, and/or inlets coupled to and/or formed in the main chassis 105.

As shown in FIG. 12, the PSA system 830 includes an air filter 908 (e.g., a filter, a high efficiency particular air (HEPA) filter, etc.). The air filter 908 is configured to receive ambient air from the air intake 904 and to filter the ambient air (e.g., by removing particulate matter and contaminants) to produce filtered air. The air filter 908 may be housed within the main chassis 105. For example, the air filter 908 may be mounted within an air intake path (e.g., a pipe, a tube, a vent, a channel, etc.) housed within the main chassis 105. In some embodiments, the air filter 908 is coupled to the air intake 904. For example, the air filter 908 may extend over the air intake 904 (e.g., over an opening of the air intake 904) and filter the ambient air as it is taken up by the IHHT device 802 via the air intake 904.

As shown in FIG. 12, the PSA system 830 includes a muffler 912 (e.g., an attenuator). The muffler 912 may be housed within the main chassis 105 and configured to receive the filtered air from the air filter 908. For example, the muffler 912 may be mounted within an air intake path (e.g., a pipe, a tube, a vent, a channel, etc.) housed within the main chassis 105. The muffler 912 may be configured to reduce intake noise and dampen pressure pulsations generated as air is drawn into the PSA system 830, thereby reducing vibration and promoting smoother airflow through the PSA system 830.

As shown in FIG. 12, the PSA system 830 includes a compressor 916. The compressor 916 may be substantially similar to the compressor 320 described with respect to FIG. 3. The compressor 916 may be housed within the main chassis 105. The compressor 916 may be configured to draw air into the IHHT device 802 such that air passes through the air intake 904, the air filter 908, and the muffler 912. The compressor 916 may be configured to receive filtered and muffled air from the muffler 912 and to increase a pressure of the filtered and muffled air to produce pressurized air. For example, the compressor 916 may increase the air pressure to a level suitable for PSA separation (e.g., approximately 1.5-3.0 bar gauge). The compressor 916 may include various subcomponents, such as a cooling fan.

As shown in FIG. 12, the PSA system 830 includes a heat exchanger 920. The heat exchanger 920 may be substantially similar to the heat exchanger 330 described with respect to FIG. 3. The heat exchanger 920 may be housed within the main chassis 105. The heat exchanger 920 may be configured to receive pressurized air from the compressor 916 and to condition the pressurized air to a proper temperature, preventing excess heat or cold from affecting the PSA separation process.

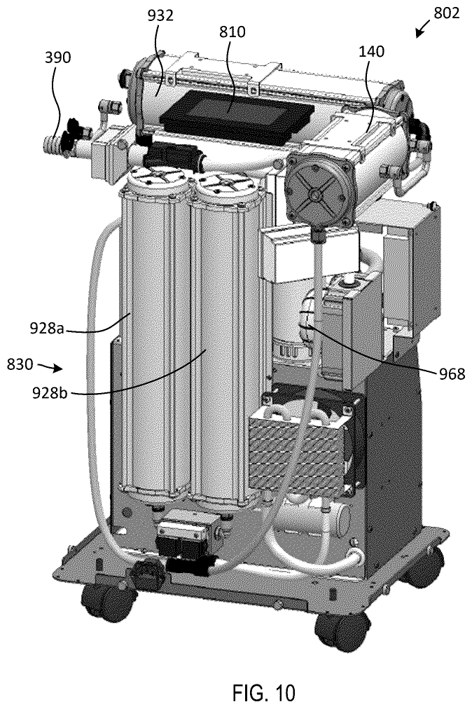

As shown in FIGS. 10-12, the PSA system 830 includes a control valve 924 and adsorptions towers 928a, 928b. The control valve 924 may be substantially similar to the central control valve assembly 340 described with respect to FIG. 3. The control valve 924 may receive the pressurized and conditioned air from the heat exchanger 920 and selectively provide the pressurized and conditioned air to adsorption tower 928a or adsorption tower 928b.

The adsorption towers 928a and 928b may be substantially similar to adsorption towers 350a and 350b. For example, the adsorption towers 928 may contain zeolite molecular sieve material that preferentially adsorbs nitrogen from compressed air. When adsorption tower 928a is in an adsorption (pressurization) phase, the control valve 924 provides the pressurized and condition air to the adsorption tower 928a and zeolite preferentially captures nitrogen. Accordingly, product gas leaving adsorption tower 928a may be an oxygen-rich (e.g., nitrogen poor, hyperoxic) gas stream having an oxygen concentration of approximately 80%-90%. Simultaneously, while adsorption tower 928a is in the adsorption phase, the adsorption tower 928b is in a desorption (depressurization/regeneration) phase, releasing trapped nitrogen to produce a nitrogen-rich (e.g., oxygen poor, hypoxic) gas stream having an oxygen concentration of approximately 8%-9%.

The adsorption towers 928a and 928b alternate roles on a timed cycle (e.g., every 5-20 seconds). The timed cycle may be determined based on control signals received by the control valve 924. For example, the control valve 924 may be signaled to direct air to adsorption tower 928a for the production of oxygen-rich gas (e.g., adsorption phase). After 15-20 seconds, the control valve 924 may be signaled to switch to directing air to adsorption tower 928b and adsorption tower 928a may enter the desorption phase. As described in greater detail herein, the control valve 924 may also be signaled to receive and direct the nitrogen-rich gas streams produced by adsorption towers 928a and 928b when they are in their respective desorption phases.

The alternating operation of adsorption towers 928a and 928b may produce an asymmetry between the nitrogen-rich gas stream and the oxygen-rich gas stream. For example, the oxygen-rich gas stream may be substantially continuous as at least one of the adsorption towers 928a, 928b is in the adsorption (pressurization) phase at any given time. The nitrogen-rich gas stream, by contrast, may be pulsatile as nitrogen-enriched gas is released from one of the adsorption towers 928a, 928b only during the desorption (depressurization) phase. While the use of two or more towers provides overlapping desorption windows that partially interleave the pulsatile output, the nitrogen-rich output remains characterized by cyclic flow rate and concentration variations that are substantially larger in magnitude and duration than the corresponding variations in the oxygen-rich output. This asymmetry in output continuity may by inherent to PSA thermodynamics where adsorption is a sustained, pressure-driven process that produces continuous product flow, while desorption is a transient, pressure-release event that produces intermittent exhaust flow. The asymmetry may influence buffering requirements of the PSA system 830 as the pulsatile nitrogen-rich stream may not be delivered directly to a breathing patient without accumulation to convert the intermittent desorption output into a continuous delivery flow, whereas the substantially continuous oxygen-rich stream can be delivered with only minor smoothing.

In some embodiments, the molecular sieve material of the adsorption towers 928 may preferentially adsorb oxygen molecules. In other embodiments, the PSA system 830 may employ vacuum pressure swing adsorption (VPSA), carbon molecular sieve (CMS)-based separation, rapid-cycle PSA with rotary valves, phased micro-column arrays, or membrane-based separation. It should be understood that any gas separation technology capable of producing both an oxygen-rich stream and a nitrogen-rich stream from ambient air may be substituted without departing from the scope of the present disclosure.

As shown in FIG. 10-12, the PSA system 830 further includes a collection chamber 932 (e.g., an equilibrium canister, an oxygen-rich product chamber, etc.). The collection chamber 932 may be configured to receive and store the oxygen-rich gas stream produced by adsorption tower 928a when adsorption tower 928a is in the adsorption phase. Similarly, the collection chamber 932 may be configured to receive and store the oxygen-rich gas stream produced by adsorption tower 928b when adsorption tower 928b is in the adsorption phase. In some embodiments, the collection chamber 932 is an equilibrium canister (e.g., equilibrium canister 360a, equilibrium canister 360b) configured to equalize pressure ripple and equalize O₂ concentration ripple coming out of the adsorptions towers 928a, 928b as described herein. The collection chamber 932 may have a storage capacity of between 200-500 mL.

As shown in FIG. 12, the PSA system 830 further includes a regulator 936 (e.g., a pressure regulator, a back-pressure regulator, a flow restricting regulator, etc.). The regulator 936 may be fluidly coupled to the collection chamber 932. The regulator 936 may be configured to maintain the oxygen-rich gas stream within a desired pressure range, thereby reducing pressure fluctuations associated with alternating operation of the adsorption towers 928a, 928b and promoting a substantially stable flow of oxygen-rich gas to downstream components of the PSA system 830. In some embodiments, the regulator 936 provides backpressure to the adsorption towers 928a, 928b to facilitate efficient PSA separation and improve consistency of oxygen concentration in the oxygen-rich gas stream.

As shown in FIG. 12, the PSA system 830 includes a hyperoxic gas path 938 (e.g., a tube, a pipe, etc.). The hyperoxic gas path 938 may be fluidly coupled to the regulator 936 and configured to receive oxygen-rich gas from the regulator 936. In various embodiments, the hyperoxic gas path 938 includes the connection between the collection chamber 932 and the regulator 936.

As shown in FIG. 12, the PSA system 830 includes a collection chamber 942 (e.g., an equilibrium canister, a hypoxic product chamber, etc.). The collection chamber 942 may be fluidly coupled to the control valve 924. The collection chamber 942 may be configured to receive nitrogen-rich gas from adsorption tower 928a when adsorption tower 928a is in the desorption phase. Similarly, the collection chamber 942 may be configured to receive nitrogen-rich gas from adsorption tower 928b when adsorption tower 928b is in the desorption phase. In some embodiments, the collection chamber 942 is an equilibrium canister as described herein. In various embodiments, the collection chamber 942 is configured to temporarily store the nitrogen-rich gas stream. The collection chamber 942 may have a storage capacity of between 200-500 mL.

In operation, the control valve 924 may fluidly couple the heat exchanger 920 to a first one of the adsorption towers 928a, 928b that is operating in the adsorption phase and may fluidly the collection chamber 942 to a second one of the adsorption towers 928a, 928b that is operating in the desorption phase. Accordingly, the control valve 924 may alternately provide pressurized and conditioned air to the adsorption tower 928a or adsorption tower 928b that is producing the oxygen-rich gas stream and may direct the nitrogen-rich gas stream released from the other adsorption tower 928a, 928b to the collection chamber 942. The collection chamber 942 may thereby accumulate nitrogen-rich gas over successive PSA cycles and reduce pulsation in the nitrogen-rich gas stream prior to downstream processing, recirculation, and/or delivery. Furthermore, the control valve 924 may be a multi-port directional control valve, rotary valve, spool valve, manifold valve assembly, and/or a plurality of coordinated valves configured to selectively couple the heat exchanger 920 and the collection chamber 942 to the adsorption towers 928a, 928b in alternating PSA cycle phases.

As shown in FIG. 12, the PSA system 830 may include a hypoxic gas path 946 (e.g., a tube, a pipe, etc.). The hypoxic gas path 946 may be fluidly coupled to the control valve 924 and the collection chamber 942. The hypoxic gas path 946 may be configured to receive nitrogen-rich gas from the control valve 924 (e.g., produced by adsorption tower 928a during desorption phase, produced by adsorption tower 928b during desorption phase) and direct the nitrogen-rich gas to the collection chamber 942.

As shown in FIG. 12, the buffering reservoir 140 is coupled to the collection chamber 942. The buffering reservoir 140 may be configured to receive, store, and/or buffer nitrogen-rich gas from the collection chamber 942. In some embodiments, the buffering reservoir 140 is a rigid pressurized vessel (e.g., aluminum or composite cylinder, or a rigid plenum) that stores the nitrogen-rich gas at a controlled pressure above ambient (e.g., approximately 0.5-2.0 bar gauge). In alternative embodiments, the buffering reservoir 140 is an accordion-geometry compliant reservoir or a semi-rigid plenum. As described with respect to FIG. 5, the buffering reservoir 140 includes the inlet check valve 504 that permits gas flow into the buffer while preventing reverse flow, the pressure relief valve 512 configured to vent excess gas to ambient atmosphere when internal pressure exceeds a predetermined threshold, and the pressure sensor 514 for closed-loop supervision by the controller 150.

As shown in FIG. 12, the IHHT device 802 includes one or more valves (e.g., a valve assembly). The one or more valves may include one or more hyperoxic delivery valves including a valve 950, a valve 954, and an electronic flow valve 958. The one or more valves may also include one or more hypoxic valves including a valve 960 and the relief valve 512. In some embodiments, the one or more valves are part of the PSA system 830. In other embodiments, the one or more valves are coupled to the PSA system 830.

As shown in FIG. 12, the valve 950 is coupled to the regulator 936 and/or the hyperoxic gas path 938. The valve 950 may be configured to selectively direct the oxygen-rich gas towards a delivery path or towards a vent path. For example, in a delivery mode, the valve 950 may direct the oxygen-rich gas to the electronic flow valve 958 for downstream mixing and delivery. In a shutoff, standby, purge, or pressure-management mode, the valve 950 may direct the oxygen-rich gas away from the electronic flow valve 958 and toward the valve 954. In various embodiments the valve 950 is a solenoid valve.

As shown in FIG. 12, the valve 954 may be fluidly coupled to the valve 950 and configured to receive oxygen-rich gas from the valve 950. The valve 954 may be a relief valve, a vent valve, a purge valve, or another pressure-release device configured to discharge oxygen-rich gas from the IHHT device 802 when a pressure condition satisfies a threshold condition. For example, the valve 954 may be actuated to vent oxygen-rich gas when an elevated pressure is detected within the collection chamber 932, the regulator 936, the hyperoxic gas path 938, and/or another upstream portion of the PSA system 830. In some embodiments, the elevated pressure is detected by one or more pressure sensors coupled to the collection chamber 932 and/or the regulator 936. Accordingly, the valve 954 may relieve excess pressure, reduce risk of overpressurization, and protect upstream PSA components during shutoff, standby, blockage, or other operating conditions in which delivery of oxygen-rich gas is reduced or interrupted.

As shown in FIG. 12, the electronic flow valve 958 may be fluidly coupled to the valve 950 and configured to receive oxygen-rich gas from the valve 950. The electronic flow valve 958 may be a metering valve, a proportional valve, a variable orifice valve, a stepper motor valve, or another electronically controllable device configured to regulate a flow rate of the oxygen-rich gas. In some embodiments, the electronic flow valve 958 controls an amount of oxygen-rich gas that is provided to a downstream delivery path for mixing with another gas stream, such as ambient air provided by a blower or the nitrogen-rich gas stream, to produce a mixed gas stream (e.g., a hyperoxic gas stream, a hypoxic gas stream) having a target oxygen concentration. Accordingly, the electronic flow valve 958 may be actuated based on one or more control signals such that an amount of opening of the electronic flow valve 958 is increased, decreased, or maintained, thereby increasing, decreasing, or maintaining the flow rate of the oxygen-rich gas to achieve a target FiO₂ for delivery to a user.

As shown in FIG. 12, the valve 960 is coupled to the buffering reservoir 140. The valve 960 may be configured to selectively provide nitrogen-rich gas from the buffering reservoir 140. For example, the valve 960 may receive control signals to open or close the valve 960, allowing or preventing nitrogen-rich gas to flow from the buffering reservoir 140.

As shown in FIG. 12, the IHHT device 802 includes a delivery path 964 (e.g., a delivery tube, a delivery pipe, etc.). The delivery path 964 may be fluidly coupled to the electronic flow valve 958 and the valve 960. The delivery path 964 may be configured to receive oxygen-rich gas from the electronic flow valve 958, nitrogen-rich gas from the valve 960, or both oxygen-rich gas from the electronic flow valve 958 and nitrogen-rich gas from the valve 960. In various embodiments, the electronic flow valve 958 is actuated to control a flow rate of oxygen-rich gas added to the delivery path 964 for mixing with the nitrogen-rich gas stream. By selectively increasing, decreasing, or maintaining the flow rate of the oxygen-rich gas introduced into the delivery path 964, the IHHT device 802 may adjust the oxygen concentration of the gas provided through the delivery path 964 to a desired target concentration or FiO₂ for delivery to a user.

As shown in FIG. 12, the IHHT device 802 includes a blower 968 (e.g., a centrifugal fan, a turbo fan, etc.). The blower 968 may be housed within the main chassis 105. The blower 968 may be fluidly coupled to the delivery path 964 and may be configured to provide an ambient air stream (e.g., a normoxic gas stream) to the delivery path 964. The ambient air stream may be drawn from the surrounding environment. The ambient air stream may have an oxygen concentration of approximately 21%. In some embodiments, the blower 968 is a turbo fan. The turbo fan may provide higher outlet pressure, more precise pressure and/or flow control, and greater resistance to downstream line resistance or pressure losses within the delivery path 964 and associated tubing, filters, valves, and patient interface components. A speed of the blower 968 (e.g., the turbo fan) may be controlled to control a flow rate and/or amount of ambient air provided to the delivery path 964. Accordingly, the blower 968 may provide a stable and controllable ambient air flow for mixing with gas produced by the adsorption towers 928a and 928b.

The blower 968 may provide the ambient air stream as a base flow and/or an auxiliary flow that is mixed with the oxygen-rich gas delivered through the electronic flow valve 958. For example, the PSA system 830 may produce an oxygen-rich gas having an oxygen concentration of approximately 80%-90% at a flow rate lower than a desired delivery flow rate (e.g., 10 liters per minute). The blower 968 may be operated to generate ambient air flow and the ambient air flow may be mixed with the oxygen-rich gas metered by the electronic flow valve 958 to produce a mixed gas stream (e.g., a hyperoxic gas stream) having the desired delivery flow rate (e.g., 40 liters per minute) and target oxygen concentration (e.g., an FiO₂ of 30%-40%). In some embodiments, the blower 968 and the electronic flow valve 958 cooperate to produce a mixed gas stream having a flow rate of approximately 30-40 L/min. In such embodiments, the blower 968 may provide the ambient air component of the mixed gas stream, while the electronic flow valve 958 adjusts the amount of oxygen-rich gas added to the ambient air to regulate the oxygen concentration.

In some embodiments, a check valve is disposed between the blower 968 and the delivery path 964. The check valve may be configured to permit flow of ambient air from the blower 968 into the delivery path 964 while substantially preventing reverse flow from the delivery path 964 toward the blower 968. The check valve may reduce or prevent backflow of oxygen-rich gas, nitrogen-rich gas, mixed gas, exhaled gas, moisture, and/or pressure pulses into the blower 968, thereby protecting the blower 968 and promoting more stable mixing, compensation, and delivery performance.

In various embodiments, the IHHT device 802 is configured to achieve a commanded inspired oxygen fraction (FiO₂) by mixing a nitrogen-rich gas stream generated by the PSA system 830 with one or more additional gas streams having an oxygen concentration greater than that of the nitrogen-rich gas stream. For example, the nitrogen-rich gas stream may be selectively mixed with ambient air from the blower 968, oxygen-rich gas from the hyperoxic gas path 938, or both ambient air and oxygen-rich gas to produce a mixed gas stream having a target oxygen concentration. In some embodiments, the target oxygen concentration is any value within a range of approximately 8%-35% O₂. The controller 150 may control the valve 960, the electronic flow valve 958, and/or the blower 968 to adjust relative amounts of the nitrogen-rich gas stream, the ambient air stream, and the oxygen-rich gas stream provided to the delivery path 964 such that the mixed gas stream attains the commanded FiO₂ for delivery to the breathing interface 160. In various embodiments, and described in further detail herein, the controller 150 receives feedback from an oxygen sensor and controls one or more components of the gas delivery system in a closed-loop manner to reduce error between the commanded FiO₂ and a measured FiO₂.

In some embodiments, the blower 968 is additionally or alternatively configured to provide flow compensation during delivery of nitrogen-rich gas. For example, the adsorption towers 928a, 928b may undergo a pressure equalization phase in which both adsorption towers 928a, 928b are in the adsorption phase and are not producing nitrogen-rich gas. During the pressure equalization phase, flow of nitrogen-rich gas toward the delivery path 964 may decrease. Accordingly, the blower 968 may be actuated synchronously with the pressure equalization phase to inject ambient air into the delivery path 964 as a flow compensation mechanism. In some embodiments, the blower 968 is controlled such that the flow compensation minimizes disruption of the target hypoxic concentration while reducing a sensation of air hunger for the user. By way of example, the blower 968 may provide a flow rate of ambient air of approximately 18 L/min. In some embodiments, the pressure equalization phase occurs for 400 milliseconds. In other embodiments, the pressure equalization phase occurs for more than 400 milliseconds. In yet other embodiments, the pressure equalization phase occurs for less than 400 milliseconds.

In various embodiments, the controller 150 is configured to control operation of the blower 968 for flow compensation by selectively determining whether a compensation burst is provided and, when provided, a duration and/or a flow rate of the compensation burst. For example, the controller 150 may cause the blower 968 to operate in an activated state to provide the compensation burst or in a deactivated state to omit the compensation burst during one or more PSA cycle phases. In some embodiments, the controller 150 causes the blower 968 to provide the compensation burst according to a selected schedule, including a fixed schedule, a programmed schedule, a per-session schedule, a per-phase schedule, a per-state schedule, or a dynamically updated schedule.

The controller 150 may be configured to adjust whether the blower 968 operates in the activated state or the deactivated state, and to further adjust a duration and/or a flow rate of the compensation burst. For example, and described in greater detail herein, the controller 150 may receive sensor data from an oxygen sensor and/or a flow sensor and may modulate the compensation burst in a closed-loop manner based on measured oxygen concentration and/or measured flow rate to regulate delivered FiO₂ and delivered flow during therapy.

Flow compensation from the blower 968 may be enabled or disabled based on a user selection entered through the user interface 810, a user selection entered through the external computing device 804, and/or a treatment protocol transmitted to the IHHT device 802 from the external computing device 804. In some embodiments, the treatment protocol includes an instruction specifying whether flow compensation is to be enabled or disabled during hypoxic delivery, hyperoxic delivery, normoxic delivery, and/or one or more transition periods therebetween. When flow compensation is enabled, the controller 150 may cause the blower 968 to operate in the activated state to inject ambient air during one or more PSA cycle phases to smooth delivered flow and improve patient comfort. When flow compensation is disabled, the controller 150 may maintain the blower 968 in the deactivated state, thereby reducing or eliminating mixing of ambient air and improving FiO₂ accuracy and/or enabling delivery of a lower oxygen concentration, such as approximately 8% O₂.

As shown in FIG. 12, the IHHT device 802 further includes an oxygen sensor 970. The oxygen sensor 970 may be coupled to the delivery path 964 and configured to receive gas (e.g., mixed gas, hyperoxic gas, hypoxic gas, normoxic gas) from the delivery path 964. The oxygen sensor 970 may include any suitable oxygen sensing device configured to measure an oxygen concentration of gas in the delivery path 964. For example, the oxygen sensor 970 may include an ultrasonic oxygen sensor, an electrochemical sensor, a galvanic sensing element, an optical sensor, a zirconia-based sensor, or another oxygen analyzer. The oxygen sensor 970 may be arranged inline with the delivery path 964, fluidly coupled to the delivery path 964, or configured to receive a sampled portion of gas from the delivery path 964. In various embodiments, and described in greater detail herein, the oxygen sensor 970 is configured to transmit sensor data (e.g., a measurement of oxygen concentration, an indication of oxygen concentration) to the controller 150 and/or the external computing device 804.

As shown in FIG. 12, the IHHT device 802 further includes a flow sensor 974. The flow sensor 974 may be coupled to the delivery path 964 and positioned downstream of the oxygen sensor 970. The flow sensor 974 may include any suitable sensing device configured to measure a flow rate of gas in the delivery path 964. For example, the flow sensor 974 may include a thermal mass flow sensor, a differential pressure-based flow sensor, a turbine flow sensor, a hot-wire or hot-film flow sensor, an ultrasonic flow sensor, a pneumotach or pneumotachograph sensor, or another gas flow analyzer. The flow sensor 974 may be arranged inline with the delivery path 964, fluidly coupled to the delivery path 964, or configured to receive a sampled portion of gas from the delivery path 964. The flow sensor 974 may be configured to transmit sensor data indicative of the gas flow rate to the controller 150 and/or the external computing device 804.

As shown in FIG. 12, the IHHT device 802 further includes a filter 978. The filter 978 may be coupled to the delivery path 964 (e.g., inline with the delivery path 964) and positioned downstream of the flow sensor 974. The filter 978 may be configured to further filter gas in the delivery path 964 prior to the gas being provided to the user.

As shown in FIG. 12, the IHHT device 802 further includes the patient output port 390. The patient output port 390 may receive filtered gas and provide the filtered gas to the breathing interface 160 for delivery to the patient. In some embodiments, the patient output port 390 receives hypoxic gas from the delivery path 964. In other embodiments, the patient output port 390 receives hyperoxic gas from the delivery path 964. In yet other embodiments, the patient output port 390 receives normoxic gas from the delivery path 964.

In some embodiments, and shown in FIG. 12, the PSA system 830 further includes a nitrogen recirculation path 980. The nitrogen recirculation path 980 path may be configured to route nitrogen-rich gas from a desorbing adsorption tower (e.g., adsorption tower 928a, adsorption tower 928b) back through adsorption towers 928a, 928b to deepen the nitrogen concentration of the nitrogen-rich gas stream. The nitrogen recirculation path 980 may be coupled to the control valve 924. The control valve 924 may selectively direct nitrogen-rich gas from the adsorption towers 928a, 928b to the collection chamber 942 for patient delivery or back to the compressor 916 via the nitrogen recirculation path 980. As shown in FIG. 12, the PSA system 830 includes a muffler 984 coupled to the nitrogen recirculation path 980 and the compressor 916. The muffler 984 may be substantially similar to the muffler 912 and may be configured to reduce intake noise and dampen pressure pulsations generated as nitrogen-rich gas is drawn back into the compressor 916.

In operation, the PSA system 830, the buffering reservoir 140, and the one or more valves including the valve 950, the valve 954, the electronic flow valve 958, and the valve 960 allow the IHHT device 802 to deliver at least two, and in various embodiments three or more, discrete gas delivery states through the patient breathing interface 160. The gas delivery states may include a hypoxic state, a hyperoxic state, and, in some embodiments, a normoxic state.

In the hypoxic state, the controller 150 may receive, from the external computing device 804, a command indicating delivery of hypoxic gas at a target oxygen concentration, such as a target oxygen concentration between approximately 8% and 17% O₂. In response to the command, the controller 150 may actuate the valve 960 to open, thereby coupling the buffering reservoir 140 to the patient output port 390 via the delivery path 964 and allowing the nitrogen-rich (e.g., oxygen poor, hypoxic) gas from the buffering reservoir 140 to flow toward the patient breathing interface 160. In some embodiments, the nitrogen-rich gas stored in the buffering reservoir 140 has an oxygen concentration of approximately 8%-9% O₂. In response to the command, the controller 150 may further actuate the valve 950 and/or the electronic flow valve 958. For example, the controller 150 may signal the electronic flow valve 958 to at least partially open such that oxygen-rich gas from the PSA system 830 (e.g., the hyperoxic gas path 938) is introduced into the delivery path 964 in a controlled amount. Accordingly, the nitrogen-rich gas from the buffering reservoir 140 and the oxygen-rich gas delivered through the electronic flow valve 958 may be mixed to produce a delivered gas stream having the commanded oxygen concentration within the hypoxic range. In some embodiments, the nitrogen-rich gas from the buffering reservoir 140 is delivered directly to the delivery path 964 without mixing with the oxygen-rich gas stream when the target oxygen concentration corresponds to the oxygen concentration of the buffering reservoir 140 (e.g., approximately 8%-9% O₂). In various embodiments, the controller 150 further controls the blower 968 according to a selected compensation mode during the hypoxic state. For example, the controller 150 may maintain the blower 968 in a deactivated state to improve FiO₂ accuracy and permit delivery of a lower hypoxic concentration, or may actuate the blower 968 to provide one or more compensation bursts during PSA phase transitions to improve flow continuity and user comfort.

In the hyperoxic state, the controller 150 may receive, from the external computing device 804, a command indicating delivery of hyperoxic gas at a target oxygen concentration. In response to the command, the controller 150 may actuate the valve 950 to direct the oxygen-rich gas from the hyperoxic gas path 938 toward the electronic flow valve 958 and may actuate the electronic flow valve 958 to open such that the oxygen-rich gas from the PSA system 830 is delivered to the delivery path 964. In some embodiments, the controller 150 causes the valve 960 to operate in a closed state such that the nitrogen-rich gas from the buffering reservoir 140 is prevented from entering the delivery path 964. In some embodiments, the oxygen-rich gas delivered through the electronic flow valve 958 has an oxygen concentration of approximately 80%-90% O₂. The controller 150 may further actuate the blower 968 to provide ambient air to the delivery path 964 such that the oxygen-rich gas and the ambient air are mixed to produce a mixed gas stream (e.g., a hyperoxic gas stream) having the commanded oxygen concentration, for example within a hyperoxic range of approximately 30%-40% O₂. In other embodiments, the controller 150 actuates the electronic flow valve 958 to deliver the oxygen-rich gas directly to the delivery path 964 without activating the blower 968, thereby providing an oxygen-rich gas with an oxygen concentration of approximately 80%-90% O₂.

In the normoxic state, the controller 150 may receive, from the external computing device 804, a command indicating delivery of normoxic gas at a target oxygen concentration, such as a target oxygen concentration of approximately 21% O₂. In response to the command, the controller 150 may cause the valve 960 to operate in a closed state to prevent nitrogen-rich gas from the buffering reservoir 140 from entering the delivery path 964 and may cause the electronic flow valve 958 to operate in a closed state, or substantially closed state, to prevent or limit delivery of oxygen-rich gas from the PSA system 830. The controller 150 may actuate the blower 968 (e.g., cause the blower to operate in an activated state) to provide ambient air to the delivery path 964 such that ambient air is delivered through the patient output port 390 to the patient breathing interface 160. In various embodiments, the PSA system 830 (e.g., the compressor 916, the adsorption towers 928a, 928b, etc.) continues operating during the normoxic state such that the oxygen-rich gas and/or the nitrogen-rich gas continue to be generated, buffered, stored, vented, or recirculated for subsequent delivery.

In some embodiments, the IHHT device 802 delivers additional gas states beyond those described above. The IHHT device 802 may support continuous or semi-continuous FiO₂ adjustment across the range of approximately 8-40% O₂, including graduated ramps between states.

The controller 150 may be configured to control the blower 968 in a closed-loop manner based on sensed operating parameters of gas delivered through the delivery path 964 and/or one or more patient-related signals. For example, the controller 150 may receive sensor data indicative of oxygen concentration from the oxygen sensor 970, sensor data indicative of gas flow from the flow sensor 974, and/or one or more signals from the sensor suite 110, and may determine, based on such data, whether to include or exclude a compensation burst and, when included, a duration and/or a flow rate of the compensation burst. The controller 150 may thereby adjust operation of the blower 968 such that delivered flow and delivered oxygen concentration more closely correspond to one or more target conditions during therapy. For example, the controller 150 may shorten the duration of the compensation burst and/or decrease the flow rate of the compensation burst in response to data from the oxygen sensor 970 indicating that measured FiO₂ is above a target FiO₂, thus indicating that excess ambient air is being added to the delivery path 964. As another example, the controller 150 may increase the duration of the compensation burst and/or increase the flow rate of the compensation burst in response to data from the flow sensor 974 indicating that delivered flow is below a target flow rate. Accordingly, the controller 150 may coordinate operation of the blower 968 with PSA cycling, gas mixing, and patient monitoring to regulate delivered flow and delivered FiO₂.

In various embodiments, operation of the blower 968 for flow compensation is additionally or alternatively adjusted based on data collected across multiple IHHT sessions. For example, the external computing device 804 may receive and store data indicative of delivered FiO₂ (e.g., from the oxygen sensor 970), delivered flow (e.g., from the flow sensor 974), patient-related signals from the sensor suite 110, and/or other treatment metrics over a plurality of treatment sessions. The external computing device 804 may analyze such data to determine updated treatment protocol parameters for a subsequent IHHT session, including whether a compensation burst is to be included or excluded, a duration of the compensation burst, and/or a flow rate of the compensation burst. The updated treatment protocol parameters may be transmitted from the external computing device 804 to the IHHT device 802 such that the controller 150 causes the blower 968 to operate according to an adaptive treatment protocol individualized to the patient across successive treatment sessions.

### Example Controller of IHHT Device

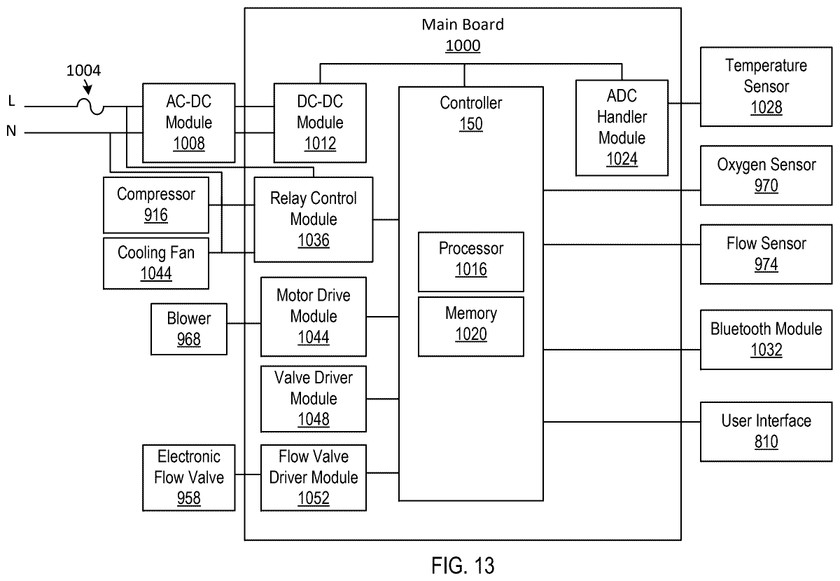

Referring now to FIG. 13, a main board 1000 (e.g., a printed circuit board (PCB), a collection of circuits, etc.) of the IHHT device 802 is shown, according to some embodiments. The main board 1000 may be configured to control operation of the IHHT device 802 to generate and deliver gas at a target flow rate, a target oxygen concentration, and for a target delivery interval. In some embodiments, the main board 1000 is a printed circuit board carrying one or more control, sensing, communication, power conversion, and actuator driver components. The main board 1000 may be housed within the chassis 105 of the IHHT device 802.

As shown in FIG. 13, input electrical power to the main board 1000 may be received from an external power source, for example an AC mains source via a line conductor (L) and a neutral conductor (N). A fuse 1004 may be disposed along an input power path and configured to interrupt current flow to the main board 1000 in the event of an overcurrent condition, thereby protecting downstream circuitry. The fuse 1004 may be a hardware component such as a cartridge fuse, a blade fuse, a surface-mount fuse, or a resettable polymeric positive temperature coefficient (PPTC) device. The input electrical power may be provided to an AC-DC module 1008 configured to convert the input AC power to a DC supply voltage. The AC-DC module 1008 may be a hardware power conversion circuit, such as a switched-mode power supply (SMPS), a linear regulator, a transformer-rectifier assembly, or another suitable AC-DC converter. In some embodiments, the AC-DC module 1008 provides one or more intermediate DC voltages (e.g., V+ and V−) to a DC-DC module 1012. The DC-DC module 1012 may be a hardware voltage regulation circuit, such as a converter (e.g., a buck converter, a boost converter, a buck-boost converter), a linear voltage regulator, or another voltage regulator configured to generate one or more regulated DC voltages for powering control electronics, sensor interface circuitry, communications circuitry, user interface circuitry, and actuator driver circuitry of the main board 1000.

As shown in FIG. 13, the main board 1000 includes the controller 150. The controller 150 may be mounted on or otherwise supported by the main board 1000. The controller 150 may include a processor 1016 and a memory 1020. The processor 1016 may be a hardware processing device, such as a microcontroller unit (MCU), a general-purpose microprocessor, a digital signal processor (DSP), an application-specific integrated circuit (ASIC), a field-programmable gate array (FPGA), or other suitable integrated circuit configured to execute instructions. The memory 1020 may be a hardware storage device including one or more of random access memory (RAM), read-only memory (ROM), flash memory, electrically erasable programmable read-only memory (EEPROM), or other non-volatile storage. The memory 1020 may store firmware, calibration data, operating parameters, control thresholds, alarm thresholds, communication parameters, and/or device configuration data. The controller 150 may be configured to receive commands (e.g., one or more treatment protocols) from the external computing device 804 and store the commands in the memory 1020 for execution by the processor 1016. In some embodiments, the controller 150 executes firmware stored in the memory 1020 to perform the control operations described herein, such that the controller 150 operates as a combination of hardware and software.

As shown in FIG. 13, the main board 1000 includes an ADC handler module 1024. The ADC handler module 1024 may include one or more analog-to-digital converter (ADC) circuits, signal conditioning amplifiers, analog multiplexers, and/or filters. The ADC handler module 1024 may be configured to receive one or more analog sensor signals, condition the analog sensor signals (e.g., amplify, filter, and/or level-shift), digitize the conditioned signals and transmit the digitized signals to the controller 150. The ADC handler module 1024 may be coupled to a temperature sensor 1028. The temperature sensor 1028 may be a hardware sensing device, such as a thermistor, a thermocouple, a resistance temperature detector (RTD), or a semiconductor-based temperature sensor (e.g., an integrated circuit temperature sensor). The temperature sensor 1028 may be configured to generate a signal representative of a temperature associated with one or more components of the IHHT device 802, such as the compressor 916, the adsorption towers 928a, 928b, or temperatures within the chassis 105. The ADC handler module 1024 may provide digital data corresponding to the temperature measured by the temperature sensor 1028 to the controller 150.

The controller 150 may be coupled to additional sensing devices, including the oxygen sensor 970 and the flow sensor 974. The oxygen sensor 970 may be configured to generate a signal indicative of the oxygen concentration of gas in the delivery path 964, and the flow sensor 974 may be configured to generate a signal indicative of a flow rate of gas in the delivery path 964. In some embodiments, the oxygen sensor 970 and the flow sensor 974 are digital sensors that provide digital output signals directly to the controller 150. In other embodiments, the oxygen sensor 970 and the flow sensor 974 provide analog outputs that are processed through the ADC handler module 1024.

As shown in FIG. 13, the controller 150 is coupled to a Bluetooth module 1032. The Bluetooth module 1032 may be a hardware wireless communication component, such as a discrete Bluetooth transceiver integrated circuit, a system-on-chip (SoC) with an integrated Bluetooth radio, a certified Bluetooth module, or a combination of a radio frequency (RF) transceiver and an antenna. The Bluetooth module 1032 may form at least a portion of the communications interface 840 described with respect to FIG. 8. The Bluetooth module 1032 may be configured to facilitate wireless communication between the IHHT device 802 and an external device, such as the external computing device 804, a mobile device, a service tool, or a monitoring system. The Bluetooth module 1032 may support one or more wireless communication protocols, including Bluetooth Classic, Bluetooth Low Energy (BLE), and/or other short-range wireless protocols. Accordingly, the controller 150 may receive commands, settings, treatment protocols, or therapy parameters from the external computing device 804 via the Bluetooth module 1032 and may transmit device status, sensor data, alerts, and operational information to the external computing device 804 via the Bluetooth module 1032.

As shown in FIG. 13, the controller 150 is coupled to the user interface 810. As described with respect to FIGS. 8 and 9, the user interface 810 may include one or more input and/or output devices, such as buttons, indicators, a display, a touchscreen, audible indicators (e.g., a buzzer or speaker), or combinations thereof. The user interface 810 may enable a user or operator to view device status, receive alerts and alarms, and provide commands or

### Settings to the Controller 150.

As shown in FIG. 13, the main board 1000 includes a relay control module 1036. The relay control module 1036 may be a hardware switching circuit, such as one or more electromechanical relays, solid-state relays (SSRs), contactors, or transistor-based switching devices. The relay control module 1036 may be configured to provide electrical power to relatively high-power loads, such as the compressor 916 and a cooling fan 1040 of the compressor 916. The cooling fan 1040 may be a hardware component, such as an axial fan, a centrifugal fan, or a blower fan, configured to dissipate heat generated by the compressor 916 during operation. In some embodiments, the relay control module 1036 receives line-derived power from the input power path (e.g., from the AC-DC module 1008 or the DC-DC module 1012) and selectively connects or disconnects such power to the compressor 916 and/or the cooling fan 1040 in response to control signals received from the controller 150. The relay control module 1036 may operate as an interface between the controller 150 and higher-power electromechanical loads (e.g., the compressor 916), enabling the controller 150 to start, stop, or cycle the compressor 916 and the cooling fan 1040.

As shown in FIG. 13, the main board 1000 includes a motor driver module 1044. The motor driver module 1044 may include one or more motor driver integrated circuits, H-bridge circuits, pulse-width modulation (PWM) controllers, or electronic speed controllers (ESCs). The motor driver module 1044 may be coupled to the blower 968. The motor driver module 1044 may be configured to receive electrical power from the DC-DC module 1012 (e.g., via the controller 150) and drive the blower 968 in response to speed, torque, duty cycle, or other command signals from the controller 150. In some embodiments, the blower 968 is a DC turbo blower or centrifugal fan, and the motor driver module 1044 provides variable-speed control to regulate the flow rate of ambient air supplied by the blower 968 to the delivery path 964. The controller 150 may provide closed-loop or open-loop control of the blower 968 based on feedback from the flow sensor 974.

As shown in FIG. 13, the main board 1000 includes a valve driver module 1048. The valve driver module 1048 may be a hardware driver circuit, such as one or more transistor switching circuits, MOSFET drivers, solenoid driver integrated circuits, or relay-based switching circuits. The valve driver module 1048 may be configured to actuate one or more on/off valves, such as solenoid valves, in response to control signals from the controller 150. For example, the valve driver module 1048 may actuate the valve 950, the valve 954, the valve 960, and/or the control valve 924. The valve driver module 1048 may provide discrete drive signals (e.g., open/close commands) to switch the respective valves between energized and de-energized states, thereby controlling the routing of hyperoxic and hypoxic gas streams within the PSA system 830 and the delivery path 964.

As shown in FIG. 13, the main board 1000 includes a flow valve driver module 1052. The flow valve driver module 1052 may include a stepper motor driver integrated circuit, a microstepping controller, a PWM generator, or a digital-to-analog converter (DAC) for analog valve control. The flow valve driver module 1052 may be coupled to the electronic flow valve 958. The flow valve driver module 1052 may be configured to actuate the electronic flow valve 958 in response to control signals from the controller 150. The electronic flow valve 958 may be a stepper motor-driven valve, a proportional valve, or another electronically controllable metering device configured to regulate a flow rate of the oxygen-rich gas. The flow valve driver module 1052 may provide a variable drive signal (e.g., a pulse-width modulated signal or a stepper motor positioning signal) to modulate the opening of the electronic flow valve 958, thereby regulating the flow rate of oxygen-rich gas delivered to the delivery path 964 and controlling the oxygen concentration of the gas delivered to the user.

During operation, the controller 150 may receive one or more commands from the external computing device 804 via the communications interface 840 (e.g., the Bluetooth module 1032) indicating a target gas delivery state, target FiO₂, target flow rate, a time interval for delivery of a particular gas state, or other operating parameter. The controller 150 may further receive sensor data from the oxygen sensor 970, the flow sensor 974, and the temperature sensor 1028. Based on the received commands and sensor data, the controller 150 may transmit control signals to the relay control module 1036, the motor driver module 1044, the valve driver module 1048, and/or the flow valve driver module 1052 to adjust operation of the compressor 916, the cooling fan 1040, the blower 968, and the various valves to regulate gas generation, buffering, mixing, and delivery.

In some embodiments, one or more functions shown in FIG. 13 may be integrated, omitted, subdivided, or arranged differently. For example, one or more sensing, communication, power conversion, and driver functions may be implemented on a common circuit board or distributed across multiple boards or modules. Likewise, one or more driver modules may be implemented as separate circuits, integrated circuits, or functions within a common control board.

The memory 1020 may store instructions that, when executed by the processor 1016, cause the controller 150 to perform one or more control operations described herein, including closed-loop concentration control, sensor monitoring, alarm generation, and gas state transitions. In some embodiments, the instructions implement one or more software modules including a running control module configured to coordinate the compressor 916, the cooling fan 1040, the control valve 924, and the PSA cycle timing; a motor control module configured to control the speed of the blower 968 and the opening of the electronic flow valve 958 based on a concentration setpoint; a valve control module configured to control switching of the valve 950, the valve 954, and the valve 960; a data collection module configured to acquire sensor data from the oxygen sensor 970, the flow sensor 974, and the temperature sensor 1028; a data storage module configured to store and retrieve operating parameters; and a communication module configured to parse and transmit commands and data via the Bluetooth module 1032. The instructions may be called within one or more interrupt service routines of the processor 1016 to ensure real-time performance of valve switching and concentration regulation.

As described above, the controller 150 may receive a command from the external computing device 804 via the communications interface 840 (e.g., the Bluetooth module 1032) indicating a target oxygen concentration (e.g., a target FiO₂) for delivery to the patient breathing interface 160. To maintain the target oxygen concentration, the controller 150 may perform a closed-loop concentration control process using feedback from the oxygen sensor 970. The controller 150 may continuously or periodically read a current oxygen concentration measurement from the oxygen sensor 970, compare the current oxygen concentration to the target oxygen concentration, and generate one or more control signals to reduce a difference between the current oxygen concentration and the target oxygen concentration.

In some embodiments, the controller 150 adjusts the oxygen concentration by controlling an opening of the electronic flow valve 958 (e.g., via the flow valve driver module 1052). When the current oxygen concentration is below the target, the controller 150 may generate a control signal to the electronic flow valve 958 (e.g., via the flow valve driver module 1052) to increase the opening, thereby increasing a flow rate of oxygen-rich gas into the delivery path 964 and raising the oxygen concentration. Conversely, when the current oxygen concentration is above the target, the controller 150 may generate a control signal to the electronic flow valve 958 (e.g., via the flow valve driver module 1052) to decrease the opening, thereby decreasing the flow rate of oxygen-rich gas and lowering the oxygen concentration. In various embodiments, the electronic flow valve 958 is a stepper motor-driven valve, and the controller 150 adjusts the opening by commanding a number of steps in a corresponding direction (e.g., open or close).

Additionally or alternatively, the controller 150 may adjust a speed of the blower 968 (e.g., via the motor driver module 1044) to regulate the oxygen concentration. For example, when the IHHT device 802 is in the hyperoxic state or the normoxic state, the controller 150 may generate a control signal to increase or decrease the speed of the blower 968 to change a flow rate of ambient air that is mixed with the oxygen-rich gas in the delivery path 964, thereby adjusting the oxygen concentration of the delivered gas. In some embodiments, the controller 150 adjusts both the opening of the electronic flow valve 958 (e.g., via the flow valve driver module 1052) and the speed of the blower 968 (e.g., via the motor driver module 1044) to achieve and maintain the target oxygen concentration while also maintaining a target total flow rate (e.g., approximately 30-40 L/min) at the patient breathing interface 160.

In some embodiments, the closed-loop control process is performed within a timer interrupt service routine of the processor 1016 to ensure real-time responsiveness. The controller 150 may sample the oxygen sensor 970 at a periodic interval (e.g., every 100 ms, every 250 ms, every 500 ms, or another suitable interval) and compute an error value representing the difference between the target oxygen concentration and the current oxygen concentration. Based on the error value, the controller 150 may generate one or more control signals to the electronic flow valve 958 (e.g., via the flow valve driver module 1052) and/or the blower 968 (e.g., via the motor driver module 1044). In some embodiments, the controller 150 applies a proportional, proportional-integral (PI), or proportional-integral-derivative (PID) control algorithm to compute the control signals from the error value. In other embodiments, the controller 150 applies a lookup table, a state machine, or another control strategy to determine the appropriate adjustments.

The controller 150 may also receive flow rate data from the flow sensor 974. The controller 150 may analyze the flow rate data in combination with the oxygen concentration data from the oxygen sensor 970 to verify that the delivered gas is being provided at both the target oxygen concentration and a target flow rate. If the flow rate falls below a minimum threshold (e.g., due to a blockage, leak, or valve malfunction), the controller 150 may generate an alarm via the user interface 810 and/or transition the IHHT device 802 to the normoxic state (e.g., by de-energizing the electronic flow valve 958 and/or the valve 960 and activating the blower 968).

The controller 150 may also monitor the temperature sensor 1028 (e.g., via the ADC handler module 1024). If a temperature associated with the compressor 916 or another component of the IHHT device 802 exceeds a temperature threshold (e.g., 50 °C), the controller 150 may generate a temperature alarm, including an audio alarm signal and a visual indication (e.g., an “Over Temperature” message on the user interface 810). In response to the temperature alarm, the controller 150 may generate a control signal to the compressor 916 and/or the cooling fan 1040 (e.g., via the relay control module 1036) to reduce a speed of the compressor 916 or increase a speed of the cooling fan 1040, and/or transition the IHHT device 802 to the normoxic state.

If the current oxygen concentration measured by the oxygen sensor 970 does not reach the target oxygen concentration within a predetermined tolerance (e.g., within ±3% of the target) for a predetermined period of time (e.g., 60 seconds), the controller 150 may determine that the target oxygen concentration cannot be achieved and generate a concentration alarm. The concentration alarm may include an audio alarm signal and/or a visual indication (e.g., a “Can't Aim” message displayed on the user interface 810). In some embodiments, the controller 150 causes the IHHT device 802 to operate in the normoxic state in response to the concentration alarm.

The controller 150 may monitor a connection status of the communications interface 840 (e.g., by monitoring the Bluetooth module 1032 for a Bluetooth connection to the external computing device 804). If the controller 150 detects a loss of communication with the external computing device 804, the controller 150 may determine that commands and safety oversight from the external computing device 804 are unavailable. In response, the controller 150 may cease delivery of nitrogen-rich gas to the valve 960 (e.g., via the valve driver module 1048) and transition the IHHT device 802 to the normoxic state by activating the blower 968 (e.g., via the motor driver module 1044) to provide ambient air through the delivery path 964 and to the patient breathing interface 160. In some embodiments, the controller 150 generates an alarm on the user interface 810 to notify the user of the communication loss.

During the closed-loop control process, the controller 150 may store operating parameters including the target oxygen concentration, interval timing values, and alarm thresholds in the memory 1020. In the event of a power loss, the controller 150 may save the current operating parameters (e.g., time and concentration values for both hypoxic and hyperoxic modes) to the memory 1020. Upon restoration of power, the controller 150 may retrieve the saved operating parameters from the memory 1020, enabling the IHHT device 802 to resume operation.

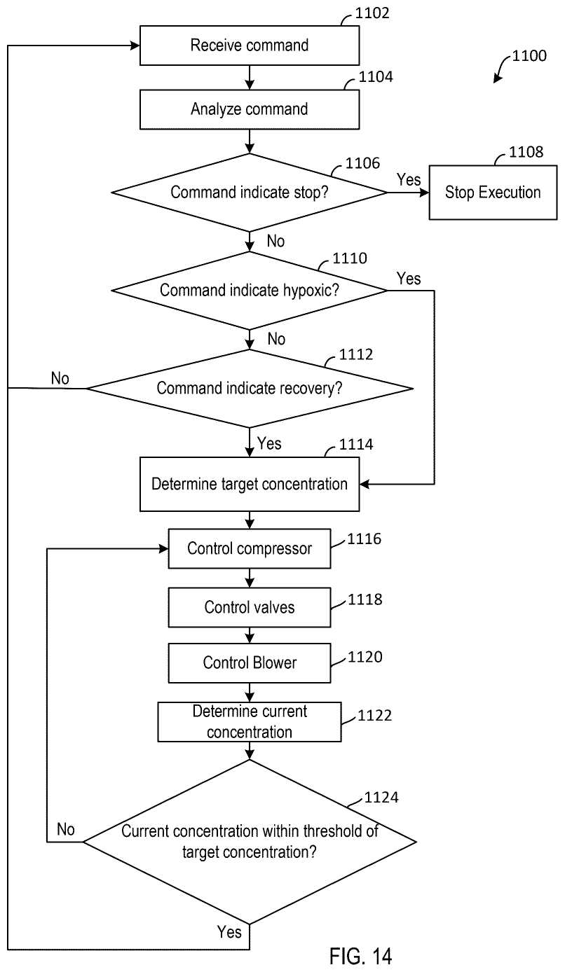

Referring now to FIG. 14, an example method 1100 for controlling gas delivery by the IHHT device 802 is shown, according to some embodiments. The method 1100 may be performed by the controller 150 of the IHHT device 802 (e.g., the processor 1016 executing instructions stored in the memory 1020).

At step 1102, the method 1100 may include receiving a command. For example, the controller 150 may receive the command from the external computing device 804 via the communications interface 840 (e.g., the Bluetooth module 1032). The command may indicate a target gas delivery state, a target oxygen concentration (e.g., a target FiO₂), a target flow rate, one or more interval durations, and/or another operating parameter as described herein.

At step 1104, the method 1100 may include analyzing the command. For example, the controller 150 may parse the command to extract an indicated gas delivery state and one or more associated parameters.

At step 1106, the method 1100 may include determining whether the command indicates a stop condition. For example, the controller 150 may determine whether the command indicates an emergency stop condition or another command indicating that execution is to stop. If it is determined by the controller 150 that the command indicates a stop condition, the method 1100 may proceed to step 1108. If it is determined by the controller 150 that the command does not indicate a stop condition, the method 1100 may proceed to step 1110.

At step 1108, the method 1100 may include stopping execution. For example, the controller 150 may cause the PSA system 830 and one or more of the valves to cease gas delivery and may stop execution of the commanded gas delivery operation.

At step 1110, the method 1100 may include determining whether the command indicates delivery of hypoxic gas. For example, the controller 150 may determine whether the gas delivery state extracted from the command corresponds to a hypoxic state. If it is determined by the controller 150 that the command indicates delivery of hypoxic gas, the method 1100 may proceed to step 1114. If it is determined by the controller 150 that the command does not indicate delivery of hypoxic gas, the method 1100 may proceed to step 1112.

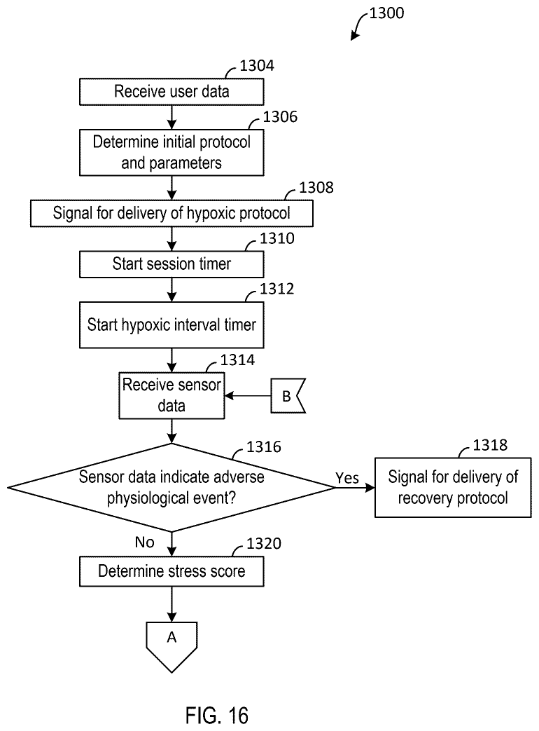

At step 1112, the method 1100 may include determining whether the command indicates recovery. For example, the controller 150 may determine whether the gas delivery state extracted from the command corresponds to a recovery state including one or more of the hyperoxic state or the normoxic state. In various embodiments, and described in greater detail herein with respect to FIGS. 16-18, the command received by the controller 150 from the external computing device 804 indicates a composition of the recovery state, such as one or more recovery sub-intervals, durations of the one or more recovery sub-intervals, and target oxygen concentrations for the one or more recovery sub-intervals. If it is determined by the controller 150 that the command indicates delivery of recovery gas, the method 1100 may proceed to step 1114. If it is determined by the controller 150 that the command does not indicate delivery of recovery gas, the method 1100 may return to step 1102 to receive a subsequent command.

At step 1114, the method 1100 may include determining a target concentration. For example, the controller 150 may determine a target oxygen concentration based on the analyzed command. In some embodiments, when the command indicates hypoxic delivery, the target oxygen concentration is within a hypoxic range (e.g., approximately 8-17% O₂). In some embodiments, when the command indicates recovery delivery, the target oxygen concentration corresponds to a recovery state (e.g., approximately 28-40% O₂ for a hyperoxic sub-interval or approximately 21% O₂ for a normoxic sub-interval).

At step 1116, the method 1100 may include controlling the compressor 916. For example, the controller 150 may transmit one or more control signals to the compressor 916 via the relay control module 1036 to start, stop, maintain, or otherwise control operation of the compressor 916. In some embodiments, the one or more control signals control operation of the compressor 916 such that the PSA system 830 continues generating one or more gas streams for delivery according to the commanded gas delivery state. In some embodiments, controlling the compressor 916 includes causing the compressor 916 to remain active during hypoxic delivery, hyperoxic delivery, and/or normoxic delivery such that gas generation by the PSA system 830 is maintained for a current interval and/or a subsequent interval. In some embodiments, the controller 150 may further control operation of the cooling fan 1040 together with operation of the compressor 916.

At step 1118, the method 1100 may include controlling one or more valves. For example, the controller 150 may transmit control signals to the valve 950, the valve 960, the electronic flow valve 958, and/or the control valve 924 (e.g., via the valve driver module 1048 and/or the flow valve driver module 1052) to configure one or more gas delivery paths for the commanded gas delivery state and to achieve the target oxygen concentration at the target flow rate. In some embodiments, controlling the one or more valves includes causing the control valve 924 to control PSA cycling of the adsorption towers 928a, 928b, causing the valve 950 to selectively direct the oxygen-rich gas stream toward the delivery path 964, causing the valve 960 to selectively permit or prevent nitrogen-rich gas from entering the delivery path 964, and causing the electronic flow valve 958 to meter a controlled amount of oxygen-rich gas into the delivery path 964. In various embodiments, the controller 150 controls the one or more valves for the commanded interval duration and adjusts one or more valve positions during the interval based on one or more measured operating parameters.

At step 1120, the method 1100 may include controlling the blower 968. For example, the controller 150 may transmit one or more control signals to the blower 968 via the motor driver module 1044 to activate the blower 968, deactivate the blower 968, and/or adjust a speed of the blower 968. In some embodiments, the controller 150 controls operation of the blower 968 such that ambient air is selectively provided to the delivery path 964 to achieve the commanded gas delivery state, the target oxygen concentration, and/or the target flow rate.

At step 1122, the method 1100 may include determining a current concentration. For example, the controller 150 may receive a current oxygen concentration measurement from the oxygen sensor 970. In some embodiments, the controller 150 also receives a current flow rate measurement from the flow sensor 974 at step 1122.

At step 1124, the method 1100 may include determining whether the current concentration is within a threshold of the target concentration. For example, the controller 150 may compare the current oxygen concentration determined at step 1122 to the target oxygen concentration determined at step 1114. If it is determined by the controller 150 that the current concentration is not within a threshold of the target concentration the method 1100 may return to step 1116 such that the controller 150 re-adjusts the compressor 916, the one or more valves, and/or the blower 968 until the target concentration is achieved. If it is determined by the controller 150 that the current concentration is within the threshold of the target concentration, the method 1100 may return to step 1102 to receive a subsequent command.

In this manner, the IHHT device 802 may continuously cycle between receiving commands, for example from the external computing device 804, and executing closed-loop hardware control to achieve and maintain a commanded gas delivery state.

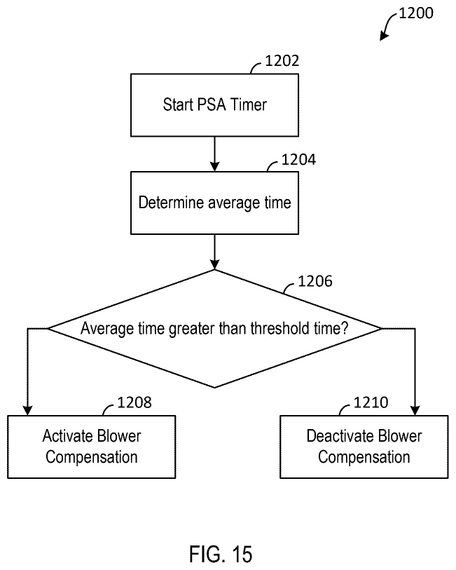

Referring now to FIG. 15, an example of a method 1200 for controlling operation of the blower 968 is shown, according to some embodiments. The method 1200 may be performed by the controller 150 of the IHHT device 802 (e.g., the processor 1016 executing instructions stored in the memory 1020). In various embodiments, the method 1200 is executed during one or more PSA phases (e.g., adsorption, desorption, equalization, etc.).

The method 1200 may begin at step 1202 with starting a PSA timer. For example, the controller 150 and/or the main board 1000 may include timing circuitry (e.g., a real time clock, a crystal oscillator, etc.) configured to track an elapsed time. The PSA timer may track the elapsed time of PSA cycles (e.g., adsorption and desorption phases).

At step 1204, the method 1200 may include determining an average time. For example, the controller 150 may determine, based on the PSA timer, an average duration associated with one or more PSA phases of the adsorption towers 928a, 928b. In some embodiments, the average time is an average duration of an adsorption phase, a pressure equalization phase, or another portion of the PSA cycle during which production and/or delivery of the nitrogen-rich gas stream is reduced, interrupted, or unavailable for delivery to the delivery path 964. The average time may be determined over a plurality of PSA cycles to reduce sensitivity to cycle-to-cycle variations and to provide a more stable basis for blower control.

At step 1206, the method 1200 may include determining whether the average time is greater than a threshold time. For example, the controller 150 may compare the average time determined at step 1204 to a threshold time stored in the memory 1020. In some embodiments, the threshold time corresponds to a duration above which interruption or reduction of the nitrogen-rich gas stream may be perceptible to a user and/or may cause a reduction in the flow rate of the hypoxic gas stream sufficient to warrant compensation by the blower 968.

If the controller 150 determines that the average time is greater than the threshold time, the method 1200 may proceed to step 1208 and activate blower compensation. For example, the controller 150 may transmit one or more control signals to the blower 968 via the motor driver module 1044 to cause the blower 968 to operate during the corresponding portion of the PSA cycle, such as during the pressure equalization phase. The blower 968 may inject ambient air into the delivery path 964 to compensate for the temporary reduction in nitrogen-rich gas flow. In some embodiments, the blower compensation is synchronized with switching of the control valve 924 and/or onset of the pressure equalization phase. In some embodiments, the blower 968 is operated at a predetermined compensation speed.

If the controller 150 determines that the average time is less than the threshold time, the method 1200 may proceed to step 1210 and deactivate blower compensation. For example, the controller 150 may transmit one or more control signals causing the blower 968 to remain off, return to an off state, or otherwise refrain from providing compensation air during the corresponding portion of the PSA cycle. In some embodiments, when the average time is below the threshold time, the reduction in nitrogen-rich gas flow is sufficiently brief that blower compensation is omitted to reduce unnecessary dilution of the hypoxic gas stream and to preserve the target oxygen concentration.

In various embodiments, the method 1200 is repeated continuously or periodically during operation of the PSA system 830 such that the controller 150 dynamically enables or disables blower compensation in response to changing PSA cycle timing. For example, as operating conditions change due to temperature, pressure, compressor performance, valve timing, or user flow demand, the average time may increase or decrease relative to the threshold time, and the controller 150 may correspondingly activate or deactivate the blower 968.

### Remote Control of IHHT Device

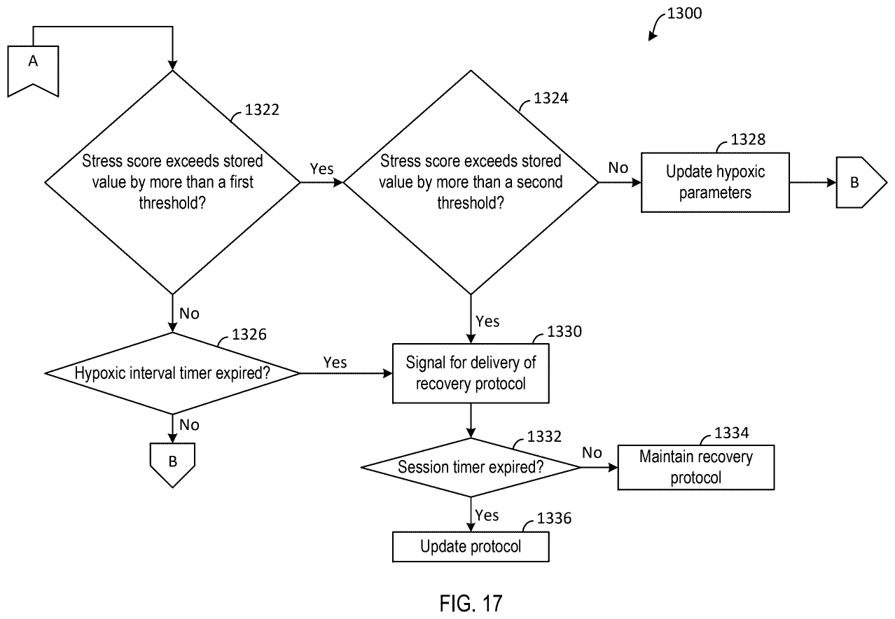

Referring now to FIGS. 16 and 17, an example of a method 1300 for controlling the IHHT device 802 is shown, according to some embodiments. The method 1300 may be performed by the external computing device 804. In various embodiments, one or more steps of the method 1300 are performed by the processing circuit 860 (e.g., the processor 864 executing instructions stored in the memory 868).

At step 1304, the method 1300 may include receiving user data. The user data may be received by the external computing device 804. The user data may be received in response to a user signing on to the treatment protocol application 240 executing on the external computing device 804 and/or providing credentials (e.g., a username, password, biometric authentication, etc.) at the external computing device 804. Alternatively or additionally, the user data may be received in response to a user providing credentials at the user interface 810 of the IHHT device 802, which may transmit the credentials to the external computing device 804 via the communications interface 840. The user data may include a user profile stored in the memory 868 or retrieved from the cloud/remote monitoring 250. The user profile may store an initial treatment protocol and initial protocol parameters including at least: a target oxygen concentration (e.g., a target FiO₂) for a hypoxic interval, a hypoxic interval duration, a session duration, and a recovery protocol composition including one or more sub-intervals having a target oxygen concentration and a duration. In some embodiments, the user profile further includes a ROS susceptibility indicator, per-user response parameters derived from prior sessions, safety thresholds (e.g., a minimum SpO₂ floor, a maximum desaturation rate, a maximum hypoxic exposure per rolling window), and clinician-configured limits. In some embodiments, the user profile is updated across sessions based on time-aligned physiological inputs, device-side measurements, and protocol metadata using a bounded adaptive estimator.

At step 1306, the method 1300 may include determining an initial treatment protocol and initial parameters for the treatment protocol. For example, the processing circuit 860 may determine the initial treatment protocol and initial parameters for the treatment protocol based on the user data. The initial treatment protocol may include a hypoxic protocol and a recovery protocol. The initial parameters for the hypoxic protocol may include a target oxygen concentration (e.g., a target FiO₂) and a hypoxic interval duration. The initial parameters for the recovery protocol may include a composition of the recovery protocol. For example, the processing circuit 860 may determine, based on the user data, that the recovery protocol includes a hyperoxic sub-interval followed by a normoxic sub-interval. The processing circuit 860 may determine a target oxygen concentration for the hyperoxic sub-interval, a duration of the hyperoxic sub-interval, and a duration of the normoxic sub-interval. The processing circuit 860 may further determine a ramp rate (e.g., a speed of transition between the hypoxic protocol and the recovery protocol, a speed of transition between sub-intervals of the recovery protocol, etc.) and a session duration based on the user data.

In some embodiments, and described above, the processing circuit 860 references the per-user response parameters and recent biomarker trends stored in the user profile to determine the initial treatment protocol and the initial parameters for the treatment protocol (e.g., initial FiO₂ targets, interval durations, ramp rates, recovery interval composition) at step 1306. In some embodiments, the processing circuit 860 applies one or more machine learning models (e.g., regression models, decision trees, random forests, support vector machines, or neural networks) pre-trained on population-level data and refined with individual historical data to determine the initial treatment protocol at step 1306.

In some embodiments, the initial treatment protocol and initial parameters for the treatment protocol are selected by the user. For example, the user may select a protocol from a list of predefined protocols using the external computing device 804 and/or the user interface 810.

At step 1308, the method 1300 may include signaling for delivery of the hypoxic protocol. For example, the external computing device 804 may transmit a command signal commanding the IHHT device 802 to deliver the hypoxic protocol. The signal may command the IHHT device 802 to deliver hypoxic gas at the initial target concentration for the hypoxic protocol. The signal may be transmitted via the communications interface 850 of the external computing device 804 to the communications interface 840 (e.g., the Bluetooth module 1032) of the IHHT device 802. In response to receiving the signal, the controller 150 of the IHHT device 802 may transmit control signals to the valves 924, 950, 960 (e.g., via the valve driver module 1048), the electronic flow valve 958 (e.g., via the flow valve driver module 1052), and/or the blower 968 (e.g., via the motor driver module 1044) to deliver gas at the commanded oxygen concentration.

At step 1310, the method 1300 may include starting a session timer. The session timer may correspond to a total session duration associated with the initial treatment protocol (e.g., 30-40 minutes). In some embodiments, the session timer is started at the IHHT device 802. For example, the controller 150 may include a timing component, such as an internal hardware timer, counter, clock peripheral, or real-time clock, configured to track elapsed time. In some embodiments, the timing component is provided on the main board 1000 as a crystal oscillator, and RTC circuit, a programmable timer, or another clock circuit communicatively coupled to the controller 150.

In some embodiments, the session timer is additionally or alternatively started at the external computing device 804. For example, the processing circuit 860 may execute instructions that run a software timer using a system clock, hardware timer, real-time clock, oscillator, or another suitable time source of the external computing device 804.

At step 1312, the method 1300 may include starting a hypoxic interval timer. The hypoxic interval timer may correspond to the hypoxic interval duration determined at step 1306. The hypoxic interval timer may be started on the IHHT device 802 and/or the external computing device 804.

At step 1314, the method 1300 may include receiving sensor data. The sensor data may be received by the external computing device 804 from one or more physiological sensors of the sensor suite 110. As described herein, the sensor suite 110 may include at least:
(i) a blood oxygen saturation (SpO₂) sensor; (ii) a heart rate variability (HRV) source derived from ECG or PPG; (iii) a blood pressure sensor; and (iv) a perfusion index sensor. Additional optional sensors include but are not limited to continuous glucose monitoring, respiration rate, end-tidal CO₂, skin temperature, ambient pressure and humidity, and accelerometer/gyroscope data. The sensor data may be transmitted from the sensor suite 110 to the external computing device 804 via the communications interface 850. The sensor data may then be analyzed by the processing circuit 860.

At step 1316, the method 1300 may include determining that the sensor data indicates an adverse physiological event. An adverse physiological event may include SpO₂ falling below a safety floor, heart rate exceeding an upper limit, blood pressure exceeding a clinician-defined stop criterion, or another condition indicating that continued hypoxic delivery poses a risk to the user. If it is determined, by the processing circuit 860, that the sensor data is indicative of an adverse physiological event (Yes at step 1316), the method 1300 may proceed to step 1318. If it is determined, by the processing circuit 860, that the sensor data does not indicate an adverse physiological event (No at step 1316), the method 1300 may proceed to step 1320

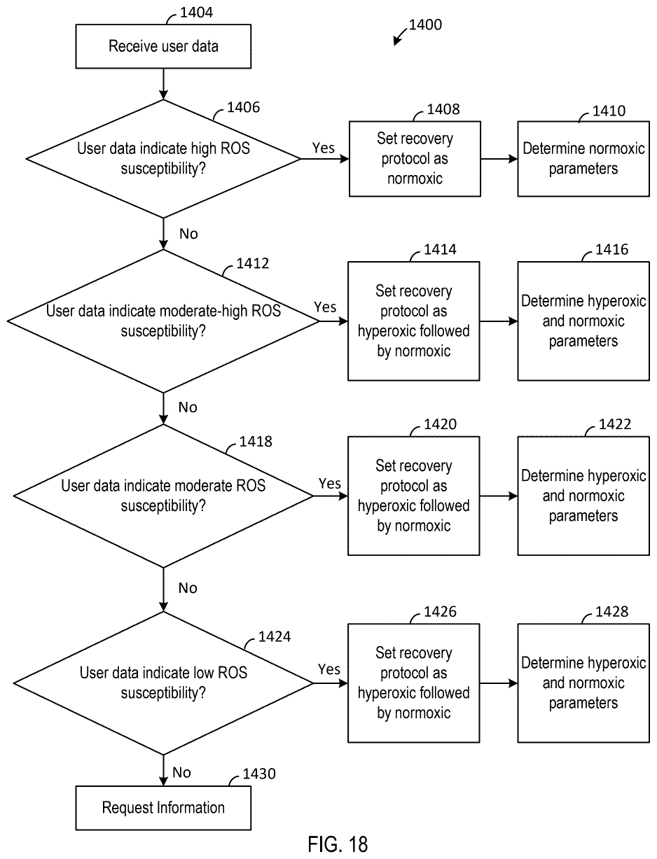

At step 1318, the method 1300 may include signaling for delivery of the recovery protocol. For example, the external computing device 804 may transmit a signal commanding the IHHT device 802 to deliver the recovery protocol. In some embodiments, the recovery protocol composition is determined based on the user's ROS susceptibility indicator as described with respect to FIG. 18. In some embodiments, the method 1300 further includes causing the user interface 810 to present an alarm (e.g., a message, an audio alarm, a visual alarm, etc.) indicative of the adverse physiological event. For example, if the SpO₂ value measured by the SpO₂ sensor of the sensor suite 110 indicates that the SpO₂ value is below 80, the external computing device 804 may cause the user interface 810 to present an SpO₂ low alarm.

At step 1320, the method 1300 may include determining a stress score (e.g., a neurovascular stress score). For example, the processing circuit 860 may determine the stress score based on the sensor data received at step 1314. The processing circuit 860 may determine the stress score by preprocessing each sensor signal to remove noise and motion artifacts; normalizing each sensor signal relative to a per-user baseline; determining features such as absolute SpO₂ value, SpO₂ rate of change, HRV time-domain metrics (e.g., RMSSD, SDNN), HRV frequency-domain metrics (e.g., LF/HF ratio), blood pressure deviation from baseline, and perfusion index trend; applying weights to the features based on the current phase of training; and fusing the weighted features using an algorithm selected from a weighted sum, a rule-based decision tree, a statistical model, or a machine learning model. The stress score may be updated at a fixed sampling interval (e.g., every 1-5 seconds).

As shown in FIG. 17, at step 1322, the method 1300 may include determining whether the stress score exceeds a stored stress score value by more than a first threshold. For example, the external computing device 804 may store a stored stress score value in the memory 868. In some embodiments, the stored stress score value is determined based on the user data, such as stress scores associated with the user profile. The stored stress score value may correspond to a target stress score for the user during hypoxic delivery. In some embodiments, the stored stress score value corresponds to a maximum stress score value for the user during hypoxic delivery. The external computing device 804 may further store one or more threshold values in the memory 868. The first threshold may correspond to a first amount by which the stress score determined at step 1320 may vary relative to the stored stress score value. Accordingly, the processing circuit 860 may compare the stress score value determined at step 1320 to the stored stress score value stored in the memory 868. If it is determined, by the processing circuit 860, that the stress score exceeds the stored stress score value by more than the first threshold (e.g., the stress score is outside of the first threshold), the method 1300 may proceed to step 1324. If it is determined, by the processing circuit 860, that the stress score does not exceed the stored stress score value by more than the first threshold (e.g., the stress score is within the first threshold), the method 1300 may proceed to step 1326.

At step 1324, the method 1300 may include determining whether the stress score exceeds the stored stress score value by more than a second threshold. The second threshold may be greater than the first threshold and may correspond to a second amount by which the stress score determined at step 1320 may vary relative to the stored stress score value. If it is determined, by the processing circuit 860, that the stress score does not exceed the stored stress score value by more than the second threshold (e.g., the stress score is within the second threshold), the method 1300 may proceed to step 1328. If it is determined, by the processing circuit 860, that the stress score exceeds the stored stress score value by more than the second threshold (e.g., the stress score is outside of the second threshold), the method 1300 may proceed to step 1330.

At step 1326, the method 1300 may include determining whether the hypoxic interval timer has expired. If it is determined, by the processing circuit 860 and/or the controller 150, that the hypoxic interval timer has not expired, the method 1300 may return to step 1314 to continue receiving sensor data. If it is determined, by the processing circuit 860 and/or the controller 150, that the hypoxic interval timer has expired, the method 1300 may proceed to step 1330.

At step 1328, the method 1300 may include updating hypoxic parameters. For example, the processing circuit 860 may command reduced hypoxic intensity (e.g., raising the target FiO₂) or a shortened hypoxic interval duration, and transmit the updated parameters to the IHHT device 802. After updating the hypoxic parameters, the method 1300 may return to step 1314 to continue receiving sensor data.

At step 1330, the method 1300 may include signaling for delivery of the recovery protocol. For example, the external computing device 804 may transmit a signal commanding the IHHT device 802 to deliver the recovery protocol, with recovery interval composition determined by the patient's ROS susceptibility profile as described with respect to FIG. 18. In various embodiments, signaling for delivery of the recovery protocol includes transmitting a target oxygen concentration and one or more interval durations for the recovery protocol.

At step 1332, the method 1300 may include determining whether the session timer has expired. If it is determined, by the processing circuit 860 and/or the controller 150, that the session timer has not expired, the method 1300 may proceed to step 1334. If it is determined, by the processing circuit 860 and/or the controller 150, that the session timer has expired, the method may proceed to step 1336.

At step 1334, the method 1300 may include maintain the recovery protocol. For example, the external computing device 804 may continue to command recovery gas until the session interval is complete.

At step 1336, the method 1300 may include updating the protocol. For example, the processing circuit 860 may update the user profile with session data including per-interval and per-session summary features. The method 1300 may then return to step 1304 to begin a new hypoxic interval with potentially updated parameters.

Referring now to FIG. 18, a method 1400 for determining recovery protocol composition based on ROS susceptibility is described, according to some embodiments. The method 1400 may be performed by the processing circuit 860 of the external computing device 804. The method 1400 may be called by the method 1300 at step 1306 when determining the initial treatment protocol and parameters and/or at step 1330 when delivery of the recovery protocol is commanded. Recovery interval parameters controlled by the external computing device 804 include at least: (a) whether to include a hyperoxic sub-interval at the beginning of the recovery interval (binary gate based on the patient's ROS susceptibility indicator); (b) if a hyperoxic sub-interval is included, the target FiO₂ (e.g., approximately 28-40%) and the duration (e.g., approximately 10-60 seconds); (c) the transition profile from the hyperoxic sub-interval to the subsequent normoxic sub-interval (e.g., step transition or graduated ramp); and (d) the duration of the normoxic sub-interval before the next hypoxic interval begins.

At step 1404, the method 1400 may include receiving user data. The user data may be received by the external computing device 804. The user data may be received in response to a user signing on to the treatment protocol application 240 executing on the external computing device 804 and/or providing credentials (e.g., a username, password, biometric authentication, etc.) at the external computing device 804. Alternatively or additionally, the user data may be received in response to a user providing credentials at the user interface 810 of the IHHT device 802, which may transmit the credentials to the external computing device 804 via the communications interface 840. The user data may include the user profile and, in particular, a reactive oxygen species (ROS) susceptibility indicator maintained in the user profile. The ROS susceptibility indicator may be based on baseline antioxidant capacity, cardiac function, comorbidity profile (e.g., diabetes mellitus, prior ischemia-reperfusion injury), age, and accumulated session history. The ROS susceptibility indicator may include a high ROS susceptibility, a moderate-high ROS susceptibility, a moderate ROS susceptibility, or a low ROS susceptibility.

At step 1406, the method 1400 may include determining whether the user data indicates a high ROS susceptibility. In some embodiments, the processing circuit 860 determines that the user data indicates a high ROS susceptibility by analyzing the ROS susceptibility indicator maintained in the user profile. In other embodiments, the processing circuit 860 determines that the user data indicates a high ROS susceptibility by analyzing the user data to derive a ROS susceptibility classification. For example, the processing circuit 860 may evaluate one or more factors stored in the user data, such as baseline antioxidant capacity, cardiac function, comorbidity profile, age, fitness level, and accumulated session history, and may determine from those factors that the user has a high ROS susceptibility. If it is determined by the processing circuit 860 that the user data indicates a high ROS susceptibility, the method 1400 may continue to step 1408. If it is determined by the processing circuit 860, that the user data does not indicate a high ROS susceptibility, the method 1400 may continue to step 1412.

At step 1408, the method 1400 may include setting the recovery protocol as normoxic. For example, the processing circuit 860 may determine that, when executing step 1306 of the method 1300, the recovery protocol is to include only a normoxic sub-interval. At step 1410, the method 1400 may include determining normoxic parameters. For example, the processing circuit 860 may determine the duration of the normoxic sub-interval. In various embodiments, the duration of the normoxic sub-interval corresponds to the remaining time of the treatment session. For example, for a patient having a high ROS susceptibility, such as a patient in early post-myocardial-infarction cardiac rehabilitation, the processing circuit 860 may cause the IHHT device 802 to transition directly to the normoxic state upon expiration of the hypoxic interval timer (step 1326 of method 1300) and/or in response to detecting an adverse physiological event (step 1316 of method 1300). The patient may then receive normoxic gas for the remainder of the treatment session (e.g., until the session timer expires).

At step 1412, the method 1400 may include determining whether the user data indicates a moderate-high ROS susceptibility. In some embodiments, the processing circuit 860 determines that the user data indicates a moderate-high ROS susceptibility by analyzing the ROS susceptibility indicator maintained in the user profile. In other embodiments, the processing circuit 860 determines that the user data indicates a moderate-high ROS susceptibility by analyzing the user data to derive a ROS susceptibility classification. For example, the processing circuit 860 may evaluate one or more factors stored in the user data, such as baseline antioxidant capacity, cardiac function, comorbidity profile, age, fitness level, recovery tolerance, and accumulated session history, and may determine from those factors that the user has a moderate-high ROS susceptibility. If it is determined by the processing circuit 860 that the user data indicates a moderate-high ROS susceptibility, the method 1400 may continue to step 1414. If it is determined by the processing circuit 860 that the user data does not indicate a moderate-high ROS susceptibility, the method 1400 may continue to step 1418.

At step 1414, the method 1400 may include setting the recovery protocol as hyperoxic followed by normoxic. For example, the processing circuit 860 may determine that, when executing step 1306 of the method 1300, the recovery protocol is to include a hyperoxic sub-interval followed by a normoxic sub-interval. At step 1416, the method 1400 may include determining hyperoxic and normoxic parameters. For example, the processing circuit 860 may determine a target oxygen concentration and a duration for the hyperoxic sub-interval and may determine a duration for the normoxic sub-interval. In various embodiments, for a patient having a moderate-high ROS susceptibility, the processing circuit 860 may determine that the hyperoxic sub-interval has a relatively short duration and a relatively low hyperoxic FiO₂ as compared to patients having moderate ROS susceptibility or low ROS susceptibility. For example, a patient having a moderate-high ROS susceptibility, such as a patient having stable heart failure with improving tolerance, may receive approximately 10 seconds of hyperoxic delivery at approximately 28% FiO₂ followed by transition to normoxic delivery.

At step 1418, the method 1400 may include determining whether the user data indicates a moderate ROS susceptibility. In some embodiments, the processing circuit 860 determines that the user data indicates a moderate ROS susceptibility by analyzing an ROS susceptibility indicator maintained in the user profile. In other embodiments, the processing circuit 860 determines that the user data indicates a moderate ROS susceptibility by analyzing the user data to derive the ROS susceptibility classification as described herein. If it is determined by the processing circuit 860 that the user data indicates a moderate ROS susceptibility, the method 1400 may continue to step 1420. If it is determined by the processing circuit 860 that the user data does not indicate a moderate ROS susceptibility, the method 1400 may continue to step 1424.

At step 1420, the method 1400 may include setting the recovery protocol as hyperoxic followed by normoxic. For example, the processing circuit 860 may determine that, when executing step 1306 of the method 1300, the recovery protocol is to include a hyperoxic sub-interval followed by a normoxic sub-interval. At step 1422, the method 1400 may include determining hyperoxic and normoxic parameters. For example, the processing circuit 860 may determine a target oxygen concentration and a duration for the hyperoxic sub-interval and may determine a duration for the normoxic sub-interval. In various embodiments, for a patient having a moderate ROS susceptibility, the processing circuit 860 may determine that the hyperoxic sub-interval has an intermediate duration and an intermediate hyperoxic FiO₂. For example, a patient having a moderate ROS susceptibility, such as a 65-year-old patient with well-controlled diabetes, may receive approximately 20 seconds of hyperoxic delivery at approximately 30% FiO₂ followed by normoxic delivery.

At step 1424, the method 1400 may include determining whether the user data indicates a low ROS susceptibility. In some embodiments, the processing circuit 860 determines that the user data indicates a low ROS susceptibility by analyzing the ROS susceptibility indicator maintained in the user profile. In other embodiments, the processing circuit 860 determines that the user data indicates a low ROS susceptibility by analyzing the user data to derive the ROS susceptibility classification as described herein. If it is determined by the processing circuit 860 that the user data indicates a low ROS susceptibility, the method 1400 may continue to step 1426. If it is determined by the processing circuit 860 that the user data does not indicate a low ROS susceptibility, the method 1400 may continue to step 1430.

At step 1426, the method 1400 may include setting the recovery protocol as hyperoxic followed by normoxic. For example, the processing circuit 860 may determine that, when executing step 1306 of the method 1300, the recovery protocol is to include a hyperoxic sub-interval followed by a normoxic sub-interval. At step 1428, the method 1400 may include determining hyperoxic and normoxic parameters. For example, the processing circuit 860 may determine a target oxygen concentration and a duration for the hyperoxic sub-interval and may determine a duration for the normoxic sub-interval. In various embodiments, for a patient having a low ROS susceptibility, the processing circuit 860 determines that the hyperoxic sub-interval has a relatively longer duration and a relatively higher hyperoxic FiO₂ as compared to patients having moderate ROS susceptibility or moderate-high ROS susceptibility. For example, a patient having a low ROS susceptibility, such as a young athlete, may receive approximately 60 seconds of hyperoxic delivery at approximately 35% FiO₂ followed by normoxic delivery before a subsequent hypoxic interval.

At step 1430, the method 1400 may include requesting additional information. For example, if the processing circuit 860 determines that the user data does not indicate any of high ROS susceptibility, moderate-high ROS susceptibility, moderate ROS susceptibility, or low ROS susceptibility with sufficient confidence, the processing circuit 860 may request additional information for classification of the user's ROS susceptibility. In some embodiments, the processing circuit 860 causes a request for information to be transmitted to a clinician system via a cloud service or remote monitoring platform, such that a clinician may review the user profile and provide an ROS susceptibility indication and/or one or more additional clinical inputs. Alternatively or additionally, the processing circuit 860 may cause a request for information to be displayed on the user interface 810 of the IHHT device 802, prompting the user and/or an operator to enter additional information. Alternatively or additionally, the processing circuit 860 may cause a request for information to be displayed on the external computing device 804, for example within the treatment protocol application 240, prompting entry of one or more missing or updated data fields. The requested information may include, for example, age, diagnosis information, recent cardiac events, comorbidity status, medication information, fitness level, recovery tolerance, prior treatment history, and/or clinician approval data. In some embodiments, until the additional information is received, the processing circuit 860 may apply a default recovery protocol, such as normoxic recovery without a hyperoxic sub-interval.

The clinical rationale for time-limited hyperoxic exposure within the recovery protocol may be based on a hormetic response to oxidative challenge. Brief hyperoxic exposure (e.g., approximately 10-20 seconds) following a hypoxic challenge may trigger beneficial antioxidant upregulation, including increased superoxide dismutase, catalase, and glutathione peroxidase activity, thereby strengthening cellular defense against subsequent oxidative challenges. This hormetic window may be narrow, such that brief exposure durations are beneficial whereas prolonged exposure durations may be harmful. The optimal hyperoxic duration may vary by patient based on baseline antioxidant capacity, cardiac function, comorbidity profile, and accumulated session history. Accordingly, the external computing device 804 may optimize the hyperoxic duration across sessions as the patient's response profile develops, including by updating the ROS susceptibility indicator and recovery protocol parameters in the user profile stored in the memory 868.

The asymmetric buffering topology of the IHHT device 802 described with respect to FIG. 12 may be advantageous for time-sequenced gas delivery and recovery control. In the IHHT device 802, the hypoxic gas stream is buffered through the buffering reservoir 140 to smooth cyclic variations associated with PSA generation before delivery to the patient, while hyperoxic and normoxic gas are delivered through separate paths that bypass the buffering reservoir 140. By buffering the hypoxic path while bypassing the buffer for hyperoxic and normoxic delivery, the IHHT device 802 may provide stable hypoxic delivery while also permitting rapid transitions into recovery delivery.

The use of separate, unbuffered hyperoxic and normoxic delivery paths may reduce transition delay between gas delivery states. For example, during a transition from hypoxic delivery to a recovery interval, the controller 150 may cease hypoxic delivery from the buffering reservoir 140, initiate delivery of a hyperoxic sub-interval through the electronic flow valve 958 and the blower 968, and thereafter transition to normoxic delivery by reducing or ceasing oxygen-rich gas delivery while maintaining ambient airflow from the blower 968. Because the recovery gas path does not traverse the buffering reservoir 140, the recovery gas may reach the patient output port 390 without waiting for displacement or purging of buffered hypoxic gas from the recovery path. In addition, because the hypoxic gas stream may remain buffered during recovery delivery, the IHHT device 802 may transition back to hypoxic delivery with reduced delay.

In some embodiments, the reduced transition delay provided by the asymmetric buffering topology may improve both control responsiveness and safety margin. For example, when a command is issued to end a hypoxic interval and begin a recovery interval, more rapid arrival of recovery gas at the breathing interface 160 may reduce continued inhalation of hypoxic gas after the transition command is issued. This may reduce overshoot between a detected physiological trigger and the patient's physiological nadir, and may permit the external computing device 804 to more precisely control hypoxic dose, recovery timing, and recovery composition during an IHHT session. Accordingly, the architecture of the IHHT device 802 may support both therapeutic precision and responsive recovery delivery, including in home-based or otherwise lightly supervised use environments.

Further, the configuration of the IHHT device 802 as a thin client under control of the external computing device 804 may offer advantages. First, it cleanly separates the SaMD (Software as a Medical Device) regulatory pathway for the treatment protocol application from the hardware device regulatory pathway for gas delivery, enabling independent software updates without requiring re-submission of the hardware device. Second, it concentrates patient data and protocol intelligence on the external computing device 804 which may be secured through existing mobile device security mechanisms (biometrics, encryption, remote wipe), rather than distributing patient data across a dedicated medical device that may be shared among patients. Third, it simplifies the hardware device by removing computational requirements for AI inference, reducing component count, power consumption, and potential failure modes within the IHHT device 802 contributing to the device simplicity and cost structure necessary for personal home deployment. The thin-client architecture is further compatible with established durable medical equipment (DME) reimbursement pathways. The IHHT device 802 shares fundamental technological heritage with home oxygen concentrators (PSA gas separation, internal compressor, single patient interface, digital control) while adding the IHHT-specific features described herein. The architectural simplicity of the IHHT device 802 is consistent with established DME device categories, enabling integration into existing home respiratory therapy reimbursement frameworks that are unavailable for higher-complexity IHHT systems confined to clinical facilities.

The methods disclosed herein may be implemented in hardware or software, or a combination of both. In one embodiment, a non-transitory machine-readable storage medium, such as the one described above, is provided, the medium comprising a data storage material encoded with machine-readable data which, when using a machine programmed with instructions for using said data, is capable of displaying any of the datasets and execution and results of this disclosure. Such data may be used for a variety of purposes, such as patient monitoring, treatment considerations, and the like. Embodiments of the methods described above may be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices in a known fashion. The computer may be, for example, a personal computer, microcomputer, or workstation of conventional design.

Each program may be implemented in a high-level procedural or object-oriented programming language to communicate with a computer system. However, the programs may be implemented in assembly or machine language, if desired. In any case, the language may be a compiled or interpreted language. Each such computer program is preferably stored on a storage medium or device (e.g., ROM or magnetic diskette) readable by a general or special-purpose programmable computer, for configuring and operating the computer when the storage medium or device is read by the computer to perform the procedures described herein. The system may also be considered to be implemented as a computer-readable storage medium, configured with a computer program, where the storage medium so configured causes a computer to operate in a specific and predefined manner to perform the functions described herein.

The signature patterns and databases thereof may be provided in a variety of media to facilitate their use. “Media” refers to a medium that contains the signature pattern information of the present disclosure. The databases of the present disclosure may be recorded on computer-readable media, e.g., any medium that may be read and accessed directly by a computer. Such media include, but are not limited to: magnetic storage media, such as floppy discs, hard disc storage media, and magnetic tape; optical storage media such as CD-ROM; electrical storage media such as RAM and ROM; and hybrids of these categories, such as magnetic/optical storage media. One skilled in the art may readily appreciate how any of the presently known computer-readable mediums may be used to create a manufacture comprising a recording of the present database information. “Recorded” refers to a process for storing information on a computer-readable medium, using any such methods as known in the art. Any convenient data storage structure may be chosen, based on the means used to access the stored information. A variety of data processor programs and formats may be used for storage, e.g., word processing text files, database format, etc.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. An intermittent hypoxia-hyperoxia training (IHHT) device, comprising:
a main chassis;
a pressure swing adsorption (PSA) system housed within the main chassis, the PSA system configured to generate a hyperoxic gas stream and a hypoxic gas stream;
a buffering reservoir housed within the main chassis and configured to store the hypoxic gas stream;
a first valve coupled to the hyperoxic gas stream;
a second valve coupled to the buffering reservoir;
a delivery path coupled to the first valve and the second valve, the delivery path including an output port; and
a blower coupled to the delivery path, the blower being operable between an activated state and a deactivated state, the blower providing an ambient air stream to the delivery path when in the activated state.

2. The IHHT device of claim 1, further comprising a controller coupled to the first valve, the second valve, and the blower.

3. The IHHT device of claim 2, wherein the controller is configured to transmit a control signal to the first valve and the second valve, the control signal causing the first valve to at least partially open and the second valve to close such that at least a portion of the hyperoxic gas stream is provided to the delivery path.

4. The IHHT device of claim 3, wherein the control signal further causes the blower to operate in the activated state such that the ambient air stream mixes with the hyperoxic gas stream to produce a mixed gas stream.

5. The IHHT device of claim 4, wherein:
the first valve is an electronic flow valve; and
the control signal further controls a flow rate of the hyperoxic gas stream, thereby controlling an oxygen concentration of the mixed gas stream.

6. The IHHT device of claim 2, wherein the controller is configured to transmit a control signal to the first valve and the second valve, the control signal causing the first valve and the second valve to at least partially open such that the hyperoxic gas stream is mixed with the hypoxic gas stream to produce a mixed gas stream at the delivery path.

7. The IHHT device of claim 6, wherein the control signal further controls operation of the first valve to control a flow rate of the hyperoxic gas stream, thereby controlling an oxygen concentration of the mixed gas stream.

8. The IHHT device of claim 7, wherein the control signal further causes the blower to operate in the deactivated state.

9. The IHHT device of claim 2, further comprising an oxygen flow sensor coupled to the delivery path and configured measure a current oxygen concentration of gas in the delivery path, wherein the controller is configured to adjust operation of one or more of the first valve, the second valve, or the blower based on the current oxygen concentration.

10. The IHHT device of claim 9, wherein the controller is configured to:
receive a command from an external computing device, the command indicating a target oxygen concentration;
compare the target oxygen concentration to the current oxygen concentration; and
adjust operation of one or more of the first valve, the second valve, or the blower based on the comparison.

11. The IHHT device of claim 1, further comprising a recirculation path coupled an adsorption tower of the PSA system and an intake of the PSA system, the recirculation path being configured to receive hypoxic gas from an outlet of the adsorption tower and direct the hypoxic gas to the intake of the PSA system.

12. A method comprising:
receiving, at a remote computing device, user data;
determining, by a processing circuit of the remote computing device, a treatment protocol based on the user data, the treatment protocol including a hypoxic protocol and a recovery protocol;
transmitting, by the remote computing device, a first signal commanding execution of the hypoxic protocol to an intermittent hypoxia-hyperoxia training (IHHT) device;
receiving, at the remote computing device, sensor data from one or more physiological sensors;
determining, by the processing circuit, an adjustment to the hypoxic protocol based on the sensor data; and
transmitting, by the remote computing device, a second signal commanding execution of the recovery protocol to the intermittent hypoxia-hyperoxia training (IHHT) device.

13. The method of claim 12, wherein determining the treatment protocol comprises determining, by the processing circuit, one or more sub-intervals of the recovery protocol and one or more target oxygen concentrations of the recovery protocol.

14. The method of claim 13, further comprising determining, by the processing circuit, a reactive oxygen species susceptibility based on the user data.

15. The method of claim 14, further comprising determining, by the processing circuit and based on the user data, that the one or more sub-intervals of the recovery protocol include one of (i) a hyperoxic interval and a normoxic interval or (ii) a normoxic interval.

16. The method of claim 12, wherein determining an adjustment to the treatment protocol comprises:
determining, by the processing circuit, a stress score based on the sensor data;
comparing, by the processing circuit, the stress score to one or more thresholds;
determining, by the processing circuit, one or more parameters of the hypoxic protocol based on the comparison; and
transmitting, from the remote computing device to the intermittent hypoxia-hyperoxia training (IHHT) device, the one or more parameters of the hypoxic protocol.

17. The method of claim 12, further comprising detecting, by the processing circuit, at least one of (i) expiration of a hypoxic interval timer or (ii) an adverse physiological event based on the sensor data, wherein, in response to detecting at least one of (i) the expiration of the hypoxic interval timer or (ii) the adverse physiological event, the remote computing device transmits the second signal.

18. A system comprising:
an intermittent hypoxia-hyperoxia training (IHHT) device, comprising:
a pressure swing adsorption (PSA) system configured to generate an oxygen-enriched gas and an oxygen-reduced gas;
a delivery path; and
a valve assembly coupled to the PSA system and the delivery path, the valve assembly configured to selectively produce a hypoxic gas stream or a hyperoxic gas stream; and
a remote computing device configured to:
determine a treatment protocol; and
transmit control signals to the IHHT device to control operation of the PSA system and the valve assembly according to the treatment protocol.

19. The system of claim 18, further comprising one or more sensors configured to transmit sensor data to the remote computing device, wherein the remote computing device is configured to adjust the treatment protocol based on the sensor data.

20. The system of claim 19, wherein the remote computing device is configured to:
transmit a first control signal to the IHHT device, the first control signal causing the valve assembly to mix the oxygen-reduced gas and the oxygen-enriched gas to produce the hypoxic gas stream, the hypoxic gas stream having a first oxygen concentration;
detect at least one of (i) expiration of a hypoxic interval timer or (ii) an adverse physiological event based on the sensor data; and
in response to detecting at least one of (i) the expiration of the hypoxic interval timer or (ii) the adverse physiological event, transmit a second control signal to the IHHT device, the second control signal causing the valve assembly to at least one of (i) mix the oxygen-reduced gas and the oxygen-enriched gas to produce the hyperoxic gas stream, the hyperoxic gas stream having a second oxygen concentration or (ii) pass a normoxic gas stream to the delivery path, the normoxic gas stream having a third oxygen concentration.
